SEQUENCE LISTING (9 sequences)

SEQ ID NO: 1 - protein, 12 aa, Artificial
  consensus sequence
LATXTVHYNPXX

SEQ ID NO: 2 - DNA, 1719 bp, Brassica napus
  CDS (1)..(1719)
  variation (271)..(273): Pro to Leu in dwf2
atgaagagggatcttcatcagttccaaggtcccaaccacgggacatcaatcgccggttcttccacttcttcccctgcggtgtttggtaaagacaagatgatgatggtcaaagaagaagaagacgacgagcttctaggagtcttgggttacaaggttaggtcttcggagatggctgaggttgcgttgaaactcgagcagcttgagacgatgatgggtaacgctcaagaagacggtttagctcacctcgcgacggatactgttcattacaaccccgctgagctttactcgtggcttgataacatgctcacggagcttaacccacccgctgcaacgaccggatctaacgctttgaacccggagattaataataataataataactcgtttttcaccggaggcgacctcaaagcgattcctggaaacgcggtttgtcgcagatctaatcagttcgcgtttgcggttgattcgtcgagtaataagcgtttgaaaccgtcctcgagccctgattcgatggttacatctccatcacctgctggagttataggaacgacggttacaaccgtgaccgagtcaactcgtcctttaatcctggtcgactcgcaggacaacggagtgcgtctagtccacgcgcttatggcctgcgctgaagccgtgcagagcagcaacttgactctagcggaggctctcgttaagcagattggtttcttggccgtctctcaagccggagccatgaggaaagtcgccacgtacttcgccgaagctctcgcgcggaggatctaccgcctctctccgccgcagacgcagatcgatcactctttatccgatactctccagatgcacttctacgagacttgcccttacctcaagttcgctcacttcacggcgaatcaggcgattctcgaggctttcgaagggaagaagagagtccacgtcatcgatttctcgatgaaccaagggcttcagtggcccgcgcttatgcaagcccttgcgttgagggaaggaggtcctccgagtttcaggttaaccggaattggtcctcccgcggcggataactccgatcatctccatgaagttggatgtaagttggctcagctcgcggaggcgattcacgtcgagtttgagtatcgtggctttgttgctaatagcttagctgatcttgatgcctcgatgcttgagcttagaccgagtgaaaccgaagctgtggcggttaactctgttttcgagctccacaagctcctaggccgtaccggtgggatagagaaagtcttcggcgttgtgaaacagattaaaccggtgattttcacggttgttgagcaagaatcgaatcataacggtccggttttcttagaccggtttactgaatcgctgcattattattcgacgttgtttgattccttggaaggtgctccgagtagccaagataaagttatgtcggaagtttatttagggaaacagatttgcaatctggtggcttgcgaaggtccggaccgtgttgagagacatgagacgctgagtcaatggtcgaaccggttcggttcgtccggttttgcgccggcgcatctcgggtctaacgcgtttaagcaagcgagtacgcttttggctttgtttaatggaggcgaaggttatcgtgtggaggagaataatgggtgtttgatgttgagttggcacactcgaccgctcataaccacctccgcttggaagctctcggcggtgcactga

SEQ ID NO: 3 - protein, 572 aa, Brassica napus
MKRDLHQFQGPNHGTSIAGSSTSSPAVFGKDKMMMVKEEEDDELLGVLGYKVRSSEMAEVALKLEQLETMMGNAQEDGLAHLATDTVHYNPAELYSWLDNMLTELNPPAATTGSNALNPEINNNNNNSFFTGGDLKAIPGNAVCRRSNQFAFAVDSSSNKRLKPSSSPDSMVTSPSPAGVIGTTVTTVTESTRPLILVDSQDNGVRLVHALMACAEAVQSSNLTLAEALVKQIGFLAVSQAGAMRKVATYFAEALARRIYRLSPPQTQIDHSLSDTLQMHFYETCPYLKFAHFTANQAILEAFEGKKRVHVIDFSMNQGLQWPALMQALALREGGPPSFRLTGIGPPAADNSDHLHEVGCKLAQLAEAIHVEFEYRGFVANSLADLDASMLELRPSETEAVAVNSVFELHKLLGRTGGIEKVFGVVKQIKPVIFTVVEQESNHNGPVFLDRFTESLHYYSTLFDSLEGAPSSQDKVMSEVYLGKQICNLVACEGPDRVERHETLSQWSNRFGSSGFAPAHLGSNAFKQASTLLALFNGGEGYRVEENNGCLMLSWHTRPLITTSAWKLSAVH

SEQ ID NO: 4 - DNA, 1722 bp, Brassica rapa
  CDS (1)..(1722)
atgaagagggatcttcatcagttccaaggtcccaaccacgggacatcaatcgccggttcttccacttcttcccctgcggtgtttggtaaagacaagatgatgatggttaaggaagaagaagacgacgagcttctaggagtcttgggttacaaggttaggtcttcggagatggctgaggttgcgttgaaactcgagcagcttgagacgatgatgggtaacgctcaagaagacggtttagctcacctcgcgacggatactgttcattacaaccccgctgagctttactcgtggcttgataacatgctcacggagcttaacccacccgctgcaacgaccgggtctaacgctttgaacccggagattaataataataataataataactcgtttttcaccggaggcgacctcaaagcgattcctggaaacgcggtttgtcgcagatctaatcagttcgcgtttgcggttgattcgtcgagtaataagcgtttgaaaccgtcctcgagccctgattcgatggttacatctccatcacctgctggagttataggaacgacggttacaaccgtgaccgagtcaactcgtcctttaatcctggtcgactcgcaggacaacggagtgcgtctagtccacgcgcttatggcctgcgctgaagccgtgcagagcagcaacttgactctagcggaggctctcgttaagcagattggtttcttagccgtctctcaagccggagccatgaggaaagtcgccacgtacttcgccgaagctctcgcgcggcggatctaccgcctctctccgccgcagacgcagatcgatcactctctatccgatactctccagatgcacttctacgagacttgcccttacctcaagttcgctcacttcacggcgaatcaggccatcctcgaggctttcgaagggaagaagagagtccacgtcatcgatttctcgatgaaccaagggcttcagtggcccgcgcttatgcaagccctcgcgttgagggaaggaggtcctccgagtttcaggttaaccggaatcggtcctcccgcggcggataactccgatcatctccacgaagttggatgtaagttggctcagctcgcggaggcgattcacgtcgagtttgagtatcgtggctttgttgctaatagcttagctgatcttgatgcttcgatgcttgagcttagaccgagtgaaaccgaagctgtggcggttaactctgttttcgagcttcacaagcttctaggccgtaccggtgggatagagaaagtcttcggcgttgtgaaacagattaaaccggtgattttcacggttgttgagcaagaatcgaatcataacggtccggttttcttagaccggtttactgaatcgctgcattattattcgacgttgtttgattccttggaaggtgctccgagtagccaagataaagtcatgtcggaagtttacttagggaaacagatttgcaatctggtggcttgcgaaggtccggaccgtgttgagagacacgagacgctgagtcagtggtcgaaccggttcggttcgtccggttttgcgccggcgcatctcgggtctaacgcgtttaagcaagcgagtacgcttttggctttgtttaatggaggcgaaggttatcgtgtggaggagaataatgggtgtttgatgttgagttggcacactcgaccgctcataaccacctccgcttggaagctctcggctgtgcactga

SEQ ID NO: 5 - protein, 573 aa, Brassica rapa
MKRDLHQFQGPNHGTSIAGSSTSSPAVFGKDKMMMVKEEEDDELLGVLGYKVRSSEMAEVALKLEQLETMMGNAQEDGLAHLATDTVHYNPAELYSWLDNMLTELNPPAATTGSNALNPEINNNNNNNSFFTGGDLKAIPGNAVCRRSNQFAFAVDSSSNKRLKPSSSPDSMVTSPSPAGVIGTTVTTVTESTRPLILVDSQDNGVRLVHALMACAEAVQSSNLTLAEALVKQIGFLAVSQAGAMRKVATYFAEALARRIYRLSPPQTQIDHSLSDTLQMHFYETCPYLKFAHFTANQAILEAFEGKKRVHVIDFSMNQGLQWPALMQALALREGGPPSFRLTGIGPPAADNSDHLHEVGCKLAQLAEAIHVEFEYRGFVANSLADLDASMLELRPSETEAVAVNSVFELHKLLGRTGGIEKVFGVVKQIKPVIFTVVEQESNHNGPVFLDRFTESLHYYSTLFDSLEGAPSSQDKVMSEVYLGKQICNLVACEGPDRVERHETLSQWSNRFGSSGFAPAHLGSNAFKQASTLLALFNGGEGYRVEENNGCLMLSWHTRPLITTSAWKLSAVH

SEQ ID NO: 6 - DNA, 1764 bp, Arabidopsis thaliana
  CDS (1)..(1764)
atgaagagagatcatcaccaattccaaggtcgattgtccaaccacgggacttcttcttcatcatcatcaatctctaaagataagatgatgatggtgaaaaaagaagaagacggtggaggtaacatggacgacgagcttctcgctgttttaggttacaaagttaggtcatcggagatggcggaggttgctttgaaactcgaacaattagagacgatgatgagtaatgttcaagaagatggtttatctcatctcgcgacggatactgttcattataatccgtcggagctttattcttggcttgataatatgctctctgagcttaatcctcctcctcttccggcgagttctaacggtttagatccggttcttccttcgccggagatttgtggttttccggcttcggattatgaccttaaagtcattcccggaaacgcgatttatcagtttccggcgattgattcttcgtcttcgtcgaataatcagaacaagcgtttgaaatcatgctcgagtcctgattctatggttacatcgacttcgacgggtacgcagattggtggagtcataggaacgacggtgacgacaaccaccacgacaacgacggcggcgggtgagtcaactcgttctgttatcctggttgactcgcaagagaacggtgttcgtttagtccacgcgcttatggcttgtgcagaagcaatccagcagaacaatttgactctagcggaagctcttgtgaagcaaatcggatgcttagctgtgtctcaagccggagctatgagaaaagtggctacttacttcgccgaagctttagcgcggcggatctaccgtctctctccgccgcagaatcagatcgatcattgtctctccgatactcttcagatgcacttttacgagacttgtccttatcttaaattcgctcacttcacggcgaaccaagcgattctcgaagcttttgaaggtaagaagagagtacacgtcattgatttctcgatgaaccaaggtcttcaatggcctgcacttatgcaagctcttgcgcttcgagaaggaggtcctccaactttccggttaaccggaattggtccaccggcgccggataattctgatcatcttcatgaagttggttgtaaattagctcagcttgcggaggcgattcacgtagaattcgaataccgtggattcgttgctaacagcttagccgatctcgatgcttcgatgcttgagcttagaccgagcgatacggaagctgttgcggtgaactctgtttttgagctacataagctcttaggtcgtcccggtgggatagagaaagttctcggcgttgtgaaacagattaaaccggtgattttcacggtggttgagcaagaatcgaaccataacggaccggttttcttagaccggtttactgaatcgttacattattattcgactctgtttgattcgttggaaggagttccgaatagtcaagacaaagtcatgtctgaagtttacttagggaaacagatttgtaatctggtggcttgtgaaggtcctgacagagtcgagagacacgaaacgttgagtcaatggggaaaccggtttggttcgtccggtttagcgccggcacatcttgggtctaacgcgtttaagcaagcgagtatgcttttgtctgtgtttaatagtggccaaggttatcgtgtggaggagagtaatggatgtttgatgttgggttggcacactcgtccactcattaccacctccgcttggaaactctcgacggcggcgtactga

SEQ ID NO: 7 - protein, 587 aa, Arabidopsis thaliana
MKRDHHQFQGRLSNHGTSSSSSSISKDKMMMVKKEEDGGGNMDDELLAVLGYKVRSSEMAEVALKLEQLETMMSNVQEDGLSHLATDTVHYNPSELYSWLDNMLSELNPPPLPASSNGLDPVLPSPEICGFPASDYDLKVIPGNAIYQFPAIDSSSSSNNQNKRLKSCSSPDSMVTSTSTGTQIGGVIGTTVTTTTTTTTAAGESTRSVILVDSQENGVRLVHALMACAEAIQQNNLTLAEALVKQIGCLAVSQAGAMRKVATYFAEALARRIYRLSPPQNQIDHCLSDTLQMHFYETCPYLKFAHFTANQAILEAFEGKKRVHVIDFSMNQGLQWPALMQALALREGGPPTFRLTGIGPPAPDNSDHLHEVGCKLAQLAEAIHVEFEYRGFVANSLADLDASMLELRPSDTEAVAVNSVFELHKLLGRPGGIEKVLGVVKQIKPVIFTVVEQESNHNGPVFLDRFTESLHYYSTLFDSLEGVPNSQDKVMSEVYLGKQICNLVACEGPDRVERHETLSQWGNRFGSSGLAPAHLGSNAFKQASMLLSVFNSGQGYRVEESNGCLMLGWHTRPLITTSAWKLSTAAY

SEQ ID NO: 8 - DNA, 1599 bp, Arabidopsis thaliana
  CDS (1)..(1599)
atgaagagagatcatcatcatcatcatcatcaagataagaagactatgatgatgaatgaagaagacgacggtaacggcatggatgagcttctagctgttcttggttacaaggttaggtcatccgaaatggctgatgttgctcagaaactcgagcagcttgaagttatgatgtctaatgttcaagaagacgatctttctcaactcgctactgagactgttcactataatccggcggagctttacacgtggcttgattctatgctcaccgaccttaatcctccgtcgtctaacgccgagtacgatcttaaagctattcccggtgacgcgattctcaatcagttcgctatcgattcggcttcttcgtctaaccaaggcggcggaggagatacgtatactacaaacaagcggttgaaatgctcaaacggcgtcgtggaaaccactacagcgacggctgagtcaactcggcatgttgtcctggttgactcgcaggagaacggtgtgcgtctcgttcacgcgcttttggcttgcgctgaagctgttcagaaagagaatctgactgtagcggaagctctggtgaagcaaatcggattcttagccgtttctcaaatcggagcgatgagaaaagtcgctacttacttcgccgaagctctcgcgcggcggatttaccgtctctctccgtcgcagagtccaatcgaccactctctctccgatactcttcagatgcacttctacgagacttgtccttatctcaagttcgctcacttcacggcgaatcaagcgattctcgaagcttttcaagggaagaaaagagttcatgtcattgatttctctatgagtcaaggtcttcaatggccggcgcttatgcaggctcttgcgcttcgacctggtggtcctcctgttttccggttaaccggaattggtccaccggcaccggataatttcgattatcttcatgaagttgggtgtaagctggctcatttagctgaggcgattcacgttgagtttgagtacagaggatttgtggctaacactttagctgatcttgatgcttcgatgcttgagcttagaccaagtgagattgaatctgttgcggttaactctgttttcgagcttcacaagctcttgggacgacctggtgcgatcgataaggttcttggtgtggtgaatcagattaaaccggagattttcactgtggttgagcaggaatcgaaccataatagtccgattttcttagatcggtttactgagtcgttgcattattactcgacgttgtttgactcgttggaaggtgtaccgagtggtcaagacaaggtcatgtcggaggtttacttgggtaaacagatctgcaacgttgtggcttgtgatggacctgaccgagttgagcgtcatgaaacgttgagtcagtggaggaaccggttcgggtctgctgggtttgcggctgcacatattggttcgaatgcgtttaagcaagcgagtatgcttttggctctgttcaacggcggtgagggttatcgggtggaggagagtgacggctgtctcatgttgggttggcacacacgaccgctcatagccacctcggcttggaaactctccaccaat

SEQ ID NO: 9 - protein, 533 aa, Arabidopsis thaliana
MKRDHHHHHHQDKKTMMMNEEDDGNGMDELLAVLGYKVRSSEMADVAQKLEQLEVMMSNVQEDDLSQLATETVHYNPAELYTWLDSMLTDLNPPSSNAEYDLKAIPGDAILNQFAIDSASSSNQGGGGDTYTTNKRLKCSNGVVETTTATAESTRHVVLVDSQENGVRLVHALLACAEAVQKENLTVAEALVKQIGFLAVSQIGAMRKVATYFAEALARRIYRLSPSQSPIDHSLSDTLQMHFYETCPYLKFAHFTANQAILEAFQGKKRVHVIDFSMSQGLQWPALMQALALRPGGPPVFRLTGIGPPAPDNFDYLHEVGCKLAHLAEAIHVEFEYRGFVANTLADLDASMLELRPSEIESVAVNSVFELHKLLGRPGAIDKVLGVVNQIKPEIFTVVEQESNHNSPIFLDRFTESLHYYSTLFDSLEGVPSGQDKVMSEVYLGKQICNVVACDGPDRVERHETLSQWRNRFGSAGFAAAHIGSNAFKQASMLLALFNGGEGYRVEESDGCLMLGWHTRPLIATSAWKLSTN

(19) United States
(12) Patent Application Publication
(10) Pub. No.: US 2014/0007297 A1
(43) Pub. Date: Jan. 2, 2014

(54) BRASSICA PLANTS WITH ALTERED ARCHITECTURE

(71) Applicants: Marc Bots, Ledeberg (BE); Benjamin Laga, Wingene (BE); Bart Den Boer, Merelbeke (BE)

(72) Inventors: Marc Bots, Ledeberg (BE); Benjamin Laga, Wingene (BE); Bart Den Boer, Merelbeke (BE)

(73) Assignee: BAYER CROPSCIENCE NV, Diegem (BE)

(21) Appl. No.: 13/695,912

(22) PCT Filed: May 2, 2011
(86) PCT No.: PCT/EP2011/002183

§ 371 Date: Nov. 2, 2012

Related U.S. Application Data
(60) Provisional application No. 61/331,057, filed on May 4, 2010.

(30) Foreign Application Priority Data
May 4, 2010 (EP) 10004680.4

(51) Int. Cl. C12N 15/82

(52) U.S. Cl. CPC C12N 15/8262
USPC 800/290

(57) ABSTRACT

The present invention relates to Brassica plants comprising at least one mutant dwarfing allele of a DELLA protein encoding gene, nucleic acid sequences representing mutant DELLA dwarfing alleles, and mutant dwarfing DELLA proteins. The invention further relates to methods for generating and identifying said plants and alleles, which can be used to obtain plants with reduced height and increased lodging resistance.

### FIELD OF THE INVENTION

This invention relates to crop plants and parts, particularly of the Brassicaceae family, in particular Brassica species, with improved agronomical characteristics, more specifically, lodging resistance. This invention also relates to DELLA proteins, more specifically repressor of gal-3 1 (RGA1) proteins, and nucleic acids encoding such DELLA proteins. More particularly, this invention relates to nucleic acids encoding mutant DELLA proteins, more specifically mutant RGA1 proteins, that reduce plant height and increase lodging resistance.

### BACKGROUND OF THE INVENTION

Lodging, i.e. flattening of standing plants by rain and/or wind, is a serious problem in many seed crops including oilseeds, because it can lead to difficulty in harvesting leading to yield loss. Lodging can be decreased by reducing plant height, and this can be accomplished by the use of plant growth regulators or the use of dwarf varieties (Muangprom et al., Molecular Breeding 17: p101-110, 2006). During the “green revolution” in the 1960s and 1970s, wheat grain yields increased substantially by the use of dwarf mutants; new varieties with altered architecture, i.e. which are shorter, have an increased grain yield at the expense of straw biomass, and are more lodging resistant, because they respond abnormally to the plant growth hormone gibberellin (GA) (Hedden, Trends Genet. 19, p5-9, 2003).

These wheat dwarf mutants were found to correspond to gain-of-function mutations in the Rht gene (Peng et al., Nature 400, p256-261, 1999), encoding a protein belonging to the DELLA protein family. DELLA proteins encoded by Rht and its orthologs in Arabidopsis (GAI, RGA, RGL1, and RGL2), maize (d8), grape (VvGAI), barley (SLN1), and rice (SLR1) have a conserved function as repressors of GA signaling and plant growth (Sun and Gubler, Ann. Rev. Plant Biol. 55, p197-223, 2004). DELLA proteins localize to the nucleus, suggesting that they act as transcriptional regulators (Silverstone et al., The Plant Cell 13, p1555-1565, 2001; Fleck and Harberd, Plant Journal 32, p935-947, 2002; Gubler et al., Plant Physiology 129, p 191-200, 2002; Itoh et al., The Plant Cell 14, p57-70, 2002; Wen and Chang, Plant Cell 14, p87-100, 2002). It has been shown that GA derepresses its signaling pathway by inducing degradation of the DELLA proteins (Gomi and Matsuoka, Current opinion in plant biology 6, p489-493, 2003)

DELLA proteins contain an N-terminal DELLA domain and a C-terminal GRAS domain. The GRAS domain is conserved among a large family of regulatory proteins, namely the GRAS family (Pysh et al., The Plant Journal 18, p111-119, 1999). This domain is likely to be the functional domain, presumably for transcriptional regulation. Additionally, the GRAS domain in the DELLA proteins was shown to be involved in F-box protein binding (Dill et al., Plant Cell 16: p1392-1405, 2004). The DELLA domain plays a role in GA-induced degradation via interaction with Arabidopsis GID1, but is not necessary for the growth-inhibiting activity of the protein (Peng et al., 1999 supra, Griffiths et al., The Plant Cell 18, 0399-3414, 2006).

It has been hypothesized that deleting the DELLA sequences turns the mutant protein into a constitutive repressor of GA signaling (Peng et al., Genes & Development 11, p3194-3205, 1997). Most gain-of-function DELLA mutations are located in the DELLA domain (see Table 1 for an overview). Deletions or specific missense mutations of the two conserved motifs (DELLA and/or VHYNP, indicated in FIG. 1) within the DELLA domain render the mutant proteins resistant to GA-induced degradation, leading to a GA-insensitive dwarf phenotype. Mutations in the C-terminal GRAS domain of DELLA proteins are generally loss-of-function and cause recessive slender phenotypes in several plant species, suggesting that this C-terminal domain is important for its repressor function (Peng et al., 1997 supra; Gubler et al., 2002 supra; Itoh et al. supra, 2002; Dill et al., 2004 supra), with some exceptions. Of the maize D9 mutant allele MUT1, the E600K mutation appeared both necessary and sufficient for the dwarf phenotype (WO 2007/124312). Also, all dwarfing mutations identified in Brassica were found to be located in the C-terminal region of the RGA1 protein. Muangprom et al., (Plant Physiology 137, p931-938, 2005) describe a GA insensitive Brassica rapa allele termed brrga1-d, which corresponds to a Q to R substitution at amino acid position 328 near the VHIID region. The B. napus semi-dominant dwarf allele bzh was found to result from a E to K substitution at amino acid position 546 (WO01/09356).

Upon breeding with the B. napus bzh dwarf mutant, difficulties appeared in the accurate determination of homozygous (dwarf; bzh/bzh) and heterozygous (semidwarf; Bzh/bzh) plants in segregating progenies due to the effect of the genetic background and the environment on the expression of this character (Foisset et al., Theor Appl Genet. 91, p756-761, 1995; Barret et al., Theor Appl Genet. 97, p828-833, 1998). Also, semi-dwarf hybrid rapeseed resulting from a cross between the bzh dwarf mutant and a normal-sized plant (“Avenir”) still display a 10% lower yield performance than that of standard varieties (http://www.international.inra.fr/layout/set/print/partnerships/with_the_private_sector/liv e_from the_labs/a_semi_dwarf_hybrid_rapeseed_that_is_promised_an_excellent_future).

When the B. rapa allele brrga1-d was crossed into B. napus, significant reductions in seed yield were observed for inbred lines homozygous for the mutant allele. Lodging resistance was significantly increased in plant homozygous for the mutant allele, but only in some of the heterozygous plants. Also, difficulties in selecting heterozygous plants during backcrossing were expected since the genetic background and environment may affect the expression of the dwarf character (Muangprom et al., 2006 supra). The effect on oil composition and glucosinolate content of the seed of these plants, the latter of which is known to be much higher in B. rapa, was not studied.

A B. napus rapid cycling dwarf has been identified (Zanewich et al., J Plant Growth Regul 10, p121-127, 1991; Frick et al., J. Amer. Soc. Hort. Sci. 119, p1137-1143, 1994), which has several undesirable pleiotropic effects (Muangprom at al., 2006 supra).

Thus, a need remains for alternative, particularly non-transgenic methods for improving lodging resistance in crop plants, particularly oilseed rape plants, without having a negative effect on the plants agronomical performance.

This invention makes a significant contribution to the art by providing Brassica plants that are resistant to lodging, while maintaining an agronomically suitable plant development. In particular, the present application discloses Brassica plants, in particular Brassica napus plants, comprising a mutant RGA1 allele in their genome which are reduced in height and lodging resistant, while maintaining normal yield levels, low glucosinolate content, and a stable dwarf phenotype that is also easily selectable in heterozygous condition. This problem is solved as herein after described in the different embodiments, examples and claims.

### SUMMARY OF THE INVENTION

In a first embodiment, the invention relates to a Brassica plant comprising in its genome at least one mutant allele of a DELLA gene, said mutant allele encoding a dwarfing mutant DELLA protein comprising the amino acid sequence of SEQ ID NO. 1, characterized in that at least one amino acid of said sequence has been modified. Further provided is a Brassica plant—wherein the at least one amino acid of SEQ ID NO. 1 that has been modified is P (proline). Preferably, the proline has been substituted by a leucine (L).

In another embodiment, the invention relates to a Brassica plant comprising a dwarfing mutant DELLA allele, wherein the dwarfing mutant DELLA protein comprising SEQ ID NO. 1 has an amino acid sequence having at least 75% sequence identity to SEQ ID NO: 3, SEQ ID NO: 5, SEQ ID NO: 7 or SEQ ID NO: 9.

The plant of the invention is more resistant to lodging and/or has a reduced height when compared to plants not comprising said mutant allele.

In another embodiment, the plant of the invention is selected from the group consisting of B. juncea, B. napus, B. rapa, B. carinata, B. oleracea and B. nigra.

Also provided are a plant cell, seed, or progeny of the plant of the invention.

The invention further relates to a Brassica seed comprising a mutant RGA 1 allele dwf2, as comprised within the seed having been deposited at the NCIMB Limited on Feb. 18, 2010, under accession number NCIMB 41697, as well as A Brassica plant, or a cell, part, seed or progeny thereof, obtained from that seed.

In yet another embodiment, the invention provides a dwarfing mutant DELLA allele encoding a dwarfing mutant DELLA protein comprising the amino acid sequence of SEQ ID NO. 1, characterized in that at least one amino acid of said sequence has been modified. Further provided is a dwarfing mutant DELLA allele, wherein the at least at least one amino acid of SEQ ID NO. 1 that has been modified is P (proline). Preferably, the proline has been substituted by a leucine (L).

In another embodiment, the invention provides a dwarfing mutant DELLA allele, wherein the dwarfing mutant DELLA protein comprising SEQ ID NO. 1 has an amino acid sequence having at least 75% sequence identity to SEQ ID NO: 3, SEQ ID NO: 5, SEQ ID NO: 7 or SEQ ID NO: 9.

The invention also provides a dwarfing mutant DELLA protein comprising the amino acid sequence of SEQ ID NO. 1, characterized in that at least one amino acid of said sequence has been modified. Further provide is a dwarfing mutant DELLA protein, wherein the at least at least one amino acid of SEQ ID NO. 1 that has been modified is P (proline). Preferably, the proline has been substituted by a leucine (L).

Further provided is a dwarfing mutant DELLA protein comprising SEQ ID NO. 1, which has an amino acid sequence having at least 75% sequence identity to SEQ ID NO: 3, SEQ ID NO: 5, SEQ ID NO: 7 or SEQ ID NO: 9.

In yet another embodiment, the invention relates to a method for transferring at least one selected dwarfing mutant DELLA allele from one plant to another plant comprising the steps of:
a. providing a first plant comprising at least one selected mutant DELLA allele as described above or generating a first plant comprising at least one selected mutant DELLA allele as described above;
b. crossing the first plant with a second plant not comprising the at least one selected mutant DELLA allele and collecting F1 hybrid seeds from the cross; and optionally the further steps of:
c. identifying F1 plants comprising the at least one selected mutant DELLA allele;
d. backcrossing F1 plants comprising the at least one selected mutant DELLA allele with the second plant not comprising the at least one selected mutant DELLA allele for at least one generation (x) and collecting BCx seeds from the crosses; and
e. identifying in every generation BCx plants comprising the at least one selected mutant DELLA allele.

The invention further relates to a method for producing a plant of the invention, comprising transferring at least one mutant DELLA allele from one plant to another plant, according to the above method. Also provided is a method to increase the lodging resistance of a plant and/or to reduce the height of a plant, comprising transferring at least one dwarfing mutant DELLA allele of the invention into the genomic DNA of said plant.

The plant of the above methods may be selected from the group consisting of B. juncea, B. napus, B. rapa, B. carinata, B. oleracea and B. nigra.

Also provided are the use of a dwarfing mutant DELLA allele of the invention to obtain a plant with reduced height or a plant with increased lodging resistance, as well as the use of the plant of the invention to produce seed comprising at least one dwarfing mutant DELLA allele, or to produce a crop of oilseed rape, comprising at least one dwarfing mutant DELLA allele.

### BRIEF DESCRIPTION OF THE DRAWINGS

FIG. 1: Multiple sequence alignment of the amino acid sequences of B. napus (bn) RGA1, B. rapa (br) RGA1, A. thaliana (at) RGA and GAI. The DELLA domain corresponds to amino acid (aa) 44-111 and the GRAS domain to aa 221-581 of the atRGA protein. Underlined are: I, conserved region I/DELLA motif; II, conserved region II/VHYNP motif; III, valine-rich region I; IV, nuclear localization signal; V, valine-rich region II/VHIID region; VI, LXXLL motif, VII, SH2-like domain. The region comprising the minimal deletion of the VHYNP motif/conserved region II that is known to confer dwarfism, based on maize d8-mpl, d8-2023 and rice SLR1-ΔTVHYNP, is boxed. The proline corresponding to the proline that has been mutated to leucine in the B. napus dwf2 mutant is in bold.

### GENERAL DEFINITIONS

The term “nucleic acid sequence” (or nucleic acid molecule or nucleotide sequence) refers to a DNA or RNA molecule in single or double stranded form, particularly a DNA encoding a protein or protein fragment according to the invention. An “endogenous nucleic acid sequence” refers to a nucleic acid sequence which is within a plant cell, e.g. an endogenous allele of a DELLA protein encoding gene present within the nuclear genome of a Brassica cell.

The term “gene” means a DNA sequence comprising a region (transcribed region), which is transcribed into an RNA molecule (e.g. a pre-mRNA, comprising intron sequences, which is then spliced into a mature mRNA) in a cell, operable linked to regulatory regions (e.g. a promoter). A gene may thus comprise several operably linked sequences, such as a promoter, a 5′ leader sequence comprising e.g. sequences involved in translation initiation, a (protein) coding region (cDNA or genomic DNA) and a 3′ non-translated sequence comprising e.g. transcription termination sites. “Endogenous gene” is used to differentiate from a “foreign gene”, “transgene” or “chimeric gene”, and refers to a gene from a plant of a certain plant genus, species or variety, which has not been introduced into that plant by transformation (i.e. it is not a ‘transgene’), but which is normally present in plants of that genus, species or variety, or which is introduced in that plant from plants of another plant genus, species or variety, in which it is normally present, by normal breeding techniques or by somatic hybridization, e.g., by protoplast fusion. Similarly, an “endogenous allele” of a gene is not an allele which is introduced into a plant or plant tissue by plant transformation, but is, for example, generated by plant mutagenesis and/or selection or obtained by screening natural populations of plants.

The terms “protein” or “polypeptide” are used interchangeably and refer to molecules consisting of a chain of amino acids, without reference to a specific mode of action, size, 3-dimensional structure or origin. A “fragment” or “portion” of a DELLA protein may thus still be referred to as a “protein”. An “isolated protein” is used to refer to a protein which is no longer in its natural environment, for example in vitro or in a recombinant bacterial or plant host cell.

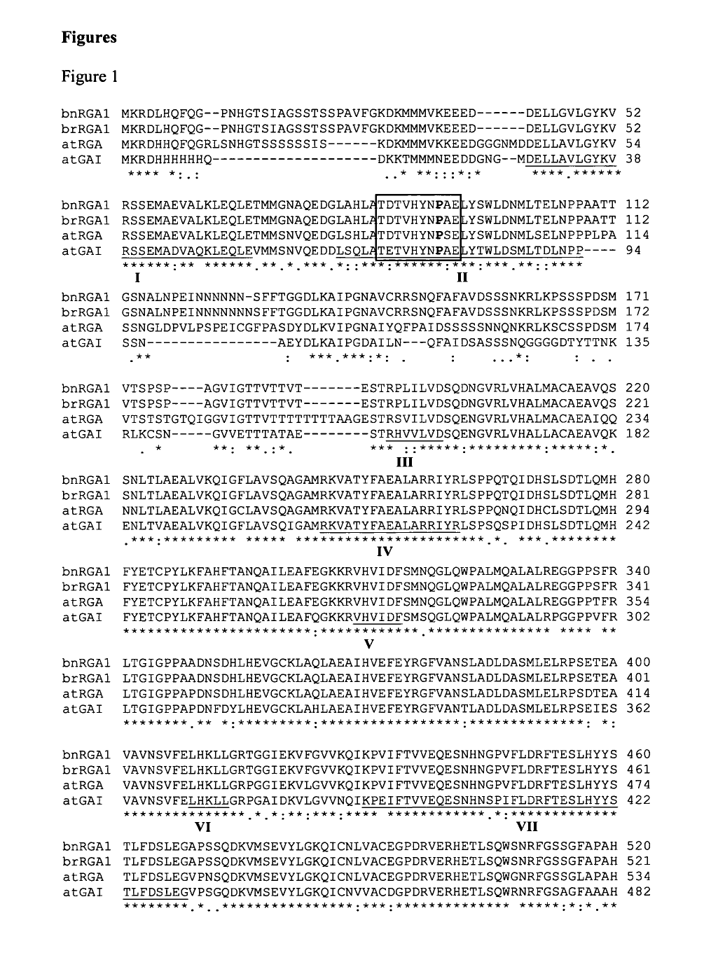

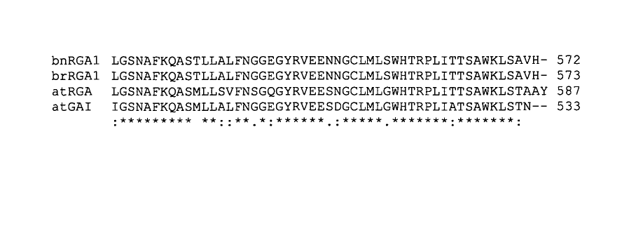

As used herein “DELLA protein”, refers to the protein(s) or polypeptide(s) with homology to the A. thaliana Repressor of gal-3 (RGA), GA-INSENSITIVE (GAI) proteins, which include but are not limited to the wheat Rht proteins, the maize d8 and D9 proteins, the rice SLENDER RICE1 (SLR1) protein, the Brassica RGA proteins (e.g. RGA1 and RGA2), the Arabidopsis RGA-LIKE1 (RGL1), RGL2, and RGL3, grapevine Vvgai and barley SLN. DELLA proteins function as nuclear repressors of plant gibberellin (GA) responses. They typically comprise an N-terminal DELLA domain (corresponding to amino acids 44-111 of the A. thaliana RGA protein represented by SEQ ID NO. 7), and a C-terminal 2/3 of the proteins which is very similar to the equivalent region of the SCARECROW (SCR) putative transcription factor from Arabidopsis, also termed the GRAS domain (corresponding to amino acids 221-581 of SEQ ID NO. 7). The DELLA domain contains two conserved regions I and II, also referred to as the DELLA and VHYNP motif (Muangprom et al., 2005 supra; Peng et al., 1999 supra; WO07/124,312). An alignment of the amino acid sequences of various DELLA proteins with indication of conserved domains is represented in FIG. 1. Corresponding domains or residues in other DELLA proteins can be determined e.g. by optimal alignment. The nucleotide sequence of the amino acid sequence of various DELLA proteins is represented in the sequence listing by SEQ ID NO: 3, SEQ ID NO: 5, SEQ ID NO: 7 and SEQ ID NO: 9. Corresponding regions, domains or residues in other DELLA sequences can be determined e.g. by optimal alignment.

DELLA proteins are localized in the nucleus where they suppress the expression of GA-responsive genes. In the presence of GA, however, DELLA proteins are targeted for breakdown. This was shown to occur by binding of GA to its receptor (GID 1 in rice and GID1a, GID1b and GID1c in Arabidopsis), which then interacts with an SCF E3 ubiquitin ligase complex to allow ubiquitination and subsequent DELLA breakdown (Djakovic-Petrovic et al., The Plant Journal 51, p117-126, 2007). The GID1-DELLA interaction specifically involves the conserved N-terminal domains I and II of the DELLA protein (Murase et al., Nature 456, p459-464, 2008), thereby explaining why mutant DELLA proteins lacking these domains confer GA-insensitivity. The formation of the GA-GID 1-DELLA complex is thought to induce a conformational change in a C-terminal GRAS domain of the DELLA protein that stimulates substrate recognition by the SCFSLY1/GID2 E3 ubiquitin ligase, proteasomic destruction of DELLA, and the consequent promotion of growth (Harberd et al., The Plant Cell 21, p1328-1339, 2009).

The term “DELLA gene” or “DELLA allele” refers herein to a nucleic acid sequence encoding a DELLA protein. The genes of all known DELLA proteins are intronless. An alignment of the nucleotide sequence of various DELLA genes/coding ssequences is represented in FIG. 2. The nucleotide sequence of various DELLA genes/coding sequences is represented in the sequence listing in SEQ ID NO: 2, SEQ ID NO: 4, SEQ ID NO: 6 and SEQ ID NO: 8.

As used herein, the term “allele(s)” means any of one or more alternative forms of a gene at a particular locus. In a diploid (or amphidiploid) cell of an organism, alleles of a given gene are located at a specific location or locus (loci plural) on a chromosome. One allele is present on each chromosome of the pair of homologous chromosomes.

As used herein, the term “homologous chromosomes” means chromosomes that contain information for the same biological features and contain the same genes at the same loci but possibly different alleles of those genes. Homologous chromosomes are chromosomes that pair during meiosis. “Non-homologous chromosomes”, representing all the biological features of an organism, form a set, and the number of sets in a cell is called ploidy. Diploid organisms contain two sets of non-homologous chromosomes, wherein each homologous chromosome is inherited from a different parent. In amphidiploid species, essentially two sets of diploid genomes exist, whereby the chromosomes of the two genomes are referred to as “homeologous chromosomes” (and similarly, the loci or genes of the two genomes are referred to as homeologous loci or genes). A diploid, or amphidiploid, plant species may comprise a large number of different alleles at a particular locus.

As used herein, the term “heterozygous” means a genetic condition existing when two different alleles reside at a specific locus, but are positioned individually on corresponding pairs of homologous chromosomes in the cell. Conversely, as used herein, the term “homozygous” means a genetic condition existing when two identical alleles reside at a specific locus, but are positioned individually on corresponding pairs of homologous chromosomes in the cell.

As used herein, the term “locus” (loci plural) means a specific place or places or a site on a chromosome where for example a gene or genetic marker is found. For example, the “RGA1 locus” refers to the position on a chromosome where the RGA1 gene (and two RGA1 alleles) may be found.

“Essentially similar”, as used herein, refers to sequences having at least 50%, at least 60%, at least 70%, at least 75%, at least 80%, at least 85%, at least 90%, at least 95%, 98%, 99% or 100% sequence identity. These nucleic acid sequences may also be referred to as being “substantially identical” or “essentially identical” to the DELLA sequences provided in the sequence listing. The “sequence identity” of two related nucleotide or amino acid sequences, expressed as a percentage, refers to the number of positions in the two optimally aligned sequences which have identical residues (x100) divided by the number of positions compared. A gap, i.e., a position in an alignment where a residue is present in one sequence but not in the other, is regarded as a position with non-identical residues. The “optimal alignment” of two sequences is found by aligning the two sequences over the entire length according to the Needleman and Wunsch global alignment algorithm (Needleman and Wunsch, 1970, J Mol Biol 48(3):443-53) in The European Molecular Biology Open Software Suite (EMBOSS, Rice et al., 2000, Trends in Genetics 16(6): 276-277; see e.g. http://www.ebi.ac.uk/emboss/align/index.html) using default settings (gap opening penalty=10 (for nucleotides)/10 (for proteins) and gap extension penalty=0.5 (for nucleotides)/0.5 (for proteins)). For nucleotides the default scoring matrix used is EDNAFULL and for proteins the default scoring matrix is EBLOSUM62.

“Stringent hybridization conditions” can be used to identify nucleotide sequences, which are substantially identical or similar to a given nucleotide sequence. Stringent conditions are sequence dependent and will be different in different circumstances. Generally, stringent conditions are selected to be about 5° C. lower than the thermal melting point (Tₘ) for the specific sequences at a defined ionic strength and pH. The Tₘ is the temperature (under defined ionic strength and pH) at which 50% of the target sequence hybridizes to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridizations (Northern blots using a probe of e.g. 100 nt) are for example those which include at least one wash in 0.2×SSC at 63° C. for 20 min, or equivalent conditions.

“High stringency conditions” can be provided, for example, by hybridization at 65° C. in an aqueous solution containing 6×SSC (20×SSC contains 3.0 M NaCl, 0.3 M Na-citrate, pH 7.0), 5×Denhardt's (100×Denhardt's contains 2% Ficoll, 2% Polyvinyl pyrollidone, 2% Bovine Serum Albumin), 0.5% sodium dodecyl sulphate (SDS), and 20 μg/ml denaturated carrier DNA (single-stranded fish sperm DNA, with an average length of 120-3000 nucleotides) as non-specific competitor. Following hybridization, high stringency washing may be done in several steps, with a final wash (about 30 min) at the hybridization temperature in 0.2-0.1×SSC, 0.1% SDS.

“Moderate stringency conditions” refers to conditions equivalent to hybridization in the above described solution but at about 60-62° C. Moderate stringency washing may be done at the hybridization temperature in 1×SSC, 0.1% SDS.

“Low stringency” refers to conditions equivalent to hybridization in the above described solution at about 50-52° C. Low stringency washing may be done at the hybridization temperature in 2×SSC, 0.1% SDS. See also Sambrook et al. (1989) and Sambrook and Russell (2001).

The term “ortholog” of a gene or protein refers herein to the homologous gene or protein found in another species, which has the same function as the gene or protein, but is (usually) diverged in sequence from the time point on when the species harboring the genes diverged (i.e. the genes evolved from a common ancestor by speciation). Orthologs of a DELLA gene, e.g. of the B. napus RGA1 gene, may thus be identified in other plant species (e.g. B. juncea, B. napus, B. rapa, B. carinata, B. oleracea and B. nigra) based on both sequence comparisons (e.g. based on percentages sequence identity over the entire sequence or over specific domains) and/or functional analysis.

The term “mutant” or “mutation” refers to e.g. a plant or allele of a gene that is different from the so-called “wild type” plant or allele/gene (also written “wildtype” or “wild-type”), which refers to a typical form of e.g. a plant or allele/gene as it most commonly occurs in nature. A “wild type plant” refers to a plant with the most common phenotype of such plant in the natural population. A “wild type allele” refers to an allele of a gene required to produce the wild-type phenotype. A mutant plant or allele can occur in the natural population or be produced by human intervention, e.g. by mutagenesis, and a “mutant allele” thus refers to an allele of a gene required to produce the mutant phenotype. As used herein, the term “mutant DELLA allele” refers to DELLA allele, which differs from its corresponding wild-type allele at one or more nucleotide positions, i.e. it comprises one or more mutations in its nucleic acid sequence when compared to the wild type allele. A mutant allele or protein may also be referred to as a variant allele or protein.

Mutations in nucleic acid sequences may include for instance:

(a) a “missense mutation”, which is a change in the nucleic acid sequence that results in the substitution of an amino acid for another amino acid;
(b) a “nonsense mutation” or “STOP codon mutation”, which is a change in the nucleic acid sequence that results in the introduction of a premature STOP codon and thus the termination of translation (resulting in a truncated protein); plant genes contain the translation stop codons “TGA” (UGA in RNA), “TAA” (UAA in RNA) and “TAG” (UAG in RNA); thus any nucleotide substitution, insertion, deletion which results in one of these codons to be in the mature mRNA being translated (in the reading frame) will terminate translation.
(c) an “insertion mutation” of one or more amino acids, due to one or more codons having been added in the coding sequence of the nucleic acid;
(d) a “deletion mutation” of one or more amino acids, due to one or more codons having been deleted in the coding sequence of the nucleic acid;
(e) a “frameshift mutation”, resulting in the nucleic acid sequence being translated in a different frame downstream of the mutation. A frameshift mutation can have various causes, such as the insertion, deletion or duplication of one or more nucleotides, but also mutations which affect pre-mRNA splicing (splice site mutations) can result in frameshifts;
(f) a “splice site mutation”, which alters or abolishes the correct splicing of the pre-mRNA sequence, resulting in a protein of different amino acid sequence than the wild type. For example, one or more exons may be skipped during RNA splicing, resulting in a protein lacking the amino acids encoded by the skipped exons. Alternatively, the reading frame may be altered through incorrect splicing, or one or more introns may be retained, or alternate splice donors or acceptors may be generated, or splicing may be initiated at an alternate position (e.g. within an intron), or alternate polyadenylation signals may be generated. Correct pre-mRNA splicing is a complex process, which can be affected by various mutations in the nucleotide sequence a genes. In higher eukaryotes, such as plants, the major spliceosome splices introns containing GU at the 5′ splice site (donor site) and AG at the 3′ splice site (acceptor site). This GU-AG rule (or GT-AG rule; see Lewin, Genes VI, Oxford University Press 1998, pp 885-920, ISBN 0198577788) is followed in about 99% of splice sites of nuclear eukaryotic genes, while introns containing other dinucleotides at the 5′ and 3′ splice site, such as GC-AG and AU-AC account for only about 1% and 0.1% respectively.

As used herein “modified”, in terms of a nucleic acid sequence or amino acid sequence, relates to one ore more mutations resulting in a deletion, insertion and/or substitution of one or more nucleic acids or amino acids in that sequence when compared to the corresponding wild-type nucleic acid or amino acid sequence.

As used herein, a “dwarfing” allele, refers to a mutant DELLA allele directing the expression of a mutant DELLA protein (a dwarfing DELLA protein) which confers a dwarf phenotype (i.e. reduced height) to the plant in which it is expressed, thereby resulting in a plant with increased lodging resistance. Such a dwarfing mutant DELLA protein comprises at least one amino acid insertion, deletion and/or substitution relative to the wild type protein, which results in the protein being not or significantly less degraded in response to GA (i.e. GA-insensitive), thereby acting as a constitutive repressor of GA induced growth. Such a mutant allele, when expressed in a plant will confer reduced responsiveness of the plant to GA-induced growth and will thereby result in a plant with reduced height, i.e. a dwarf plant, and/or a plant with increased lodging resistance. Basically, any mutation which results in a protein comprising at least one amino acid insertion, deletion and/or substitution relative to the wild type protein can lead to a dwarfing mutant DELLA protein. It is, however, understood that mutations in certain parts of the protein encoding sequence are more likely to result in a dwarfing DELLA allele, such as mutations in DNA regions encoding conserved domains like the DELLA domain (comprising the DELLA motif, spacer region, i.e. the region between the DELLA and VHYNP, and VHYNP motif).

A “dwf2 mutation” or “dwf2 mutant allele”, as used herein, refers to a mutation in a DELLA allele that leads to a substitution in the encoded DELLA protein of the proline corresponding to P91 of the B. napus RGA1 amino acid sequence (SEQ ID NO. 3) to another amino acid, preferably leucine (L). In such a dwf2 mutant allele, the codon corresponding to nucleotides (nt) 271-273 of the B. napus RGA1 genomic DNA/coding sequence (SEQ ID NO. 2) has been altered such that it does not encode a proline anymore but another amino acid, preferably leucine (e.g. CCC mutated to CTC). Determining the corresponding amino acids or nucleotide positions in another sequence can be done by methods known in the art such as optimal alignment, as described above.

“Gibberellins” or “GAs” are plant hormones that regulate growth and influence various developmental processes, including stem elongation, germination, dormancy, flowering, sex expression, enzyme induction, and leaf and fruit senescence. GAs are diterpenoid acids that are synthesized by the terpenoid pathway in plastids and then modified in the endoplasmic reticulum and cytosol until they reach their biologically-active form. Gibberellic acid, which was the first gibberellin to be structurally characterized, is known as GA3.

By “dwarf plant” is intended to mean an atypically small plant. Generally, such a “dwarf plant” has an altered architecture in that it has a stature or height that is reduced from that of a typical plant by about 5%, 10%, 15%, 20%, 25%, 30%, 35%, 40%, 45%, 50%, 55%, 60% or greater. Generally, but not exclusively, such a dwarf plant is characterized by a reduced stem, stalk or trunk length when compared to the typical plant. Advantages of dwarf plants include the possibility of very early sowing; no need for spraying growth regulators due to less stem elongation before winter; better frost tolerance; ease of monitoring of the crop due to a shorter size which facilitates plant-protection treatments; increased lodging resistance; ease of harvesting leading to less harvest loss and increased yield.

The term “lodging” as used herein, refers to flattening of standing plants by rain and/or wind whereby the crop or pods falls below cutter level at harvest. Lodging typically leads to difficulties in harvesting and harvest loss/yield loss. “Lodging resistance” thus refers to plants being less prone to lodging than a typical plant. Thus, “increased lodging resistance” or “reduced lodging” as used herein, refers to plants being less affected by lodging than a typical plant. Lodging resistance can for instance be evaluated by determining the ratio of undisturbed plant height to straightened plant height, as e.g. described by Muangprom et al. (1996) or e.g. as described below on a scale of 1 to 9. A lodging resistant plant has a lodging resistance that is increased or a lodging that is reduced from that of a typical plant by about 5%, 10%, 20%, 30%, 40%, 50%, 60%, 70%, 80%, 90% or greater. Plant with inceased lodging resistance display less harvesting difficulties and thus less harvest loss than plants with a lower lodging resistance, thereby improving the overall yield. Increased lodging resistance can result from a reduced height or stature. As used herein, “reduced height” of a plant refers to a stature or height that is reduced from that of a typical plant by about 5%, 10%, 15%, 20%, 25%, 30%, 35%, 40%, 45%, 50%, 55%, 60% or greater.

The term “mutant DELLA protein”, as used herein, e.g. a mutant RGA protein, refers to a protein encoded by a mutant DELLA nucleic acid sequence (“DELLA allele” or “DELLA gene”) whereby the mutation results in a change in the amino acid sequence of the protein when compared to the wild-type protein. A “dwarfing DELLA protein”, is a mutant DELLA protein which, when expressed in a plant, will result in a plant with reduced height (i.e. a dwarf plant) and/or increased lodging resistance when compared to a plant not expressing that protein. Typically, in such a dwarfing DELLA protein amino acids or amino acid domains essential to the protein's ability to be degraded in response to GA have been substituted, deleted or disrupted, thus making the protein GA-insensitive. Such a dwarfing DELLA protein still acts as a growth repressor. Thus, the mutation causing the DELLA protein of the invention to confer a dwarf phenotype is a gain-of-function mutation, whereby the mutant DELLA protein acts as a constitutive growth repressor. A mutant DELLA protein of the invention does not include a DELLA protein with a loss of function mutation, as such mutations will cause an increased plant height due to loss of DELLA repressor function. A mutant DELLA protein is encoded by a mutant DELLA allele or gene.

Examples of mutant dwarfing DELLA alleles/proteins are known in the art and an overview of such mutants in presented in table 1. The dwarfing effect of these mutations was confirmed by expression of mutant GAI proteins carrying corresponding mutations in Arabidopsis (Willige et al., The Plant Cell 19, p1209-1220, 2007).

TABLE 1
Overview of mutant DELLA proteins conferring a dwarf phenotype and their
references, which are all incorporated herein by reference.

| Species | mutant | | |
|---|---|---|---|
| gene | name | mutation | reference |
| Z. mais | | | |
| d8 | D8-Mpl | Δ1-105 | Peng et al., 1999 supra |
| | D8-1 | D55G, Δ56-59 | Peng et al., 1999 supra |
| | D8-2023 | Δ87-98 | Peng et al., 1999 supra |
| D9 | MUT1 | N11S, R15M, | WO 07/124312 |
| | | A108T, G427D, | |
| | | INDEL511-525, | |
| | | E600K | |
| O. sativa | | | |
| SLR1 | ΔDELLA | Δ39-55 | Itoh et al., 2002 supra |
| | Δspace | Δ69-80 | Itoh et al., 2002 supra |
| | ΔTVHYNP | Δ87-104 | Itoh et al., 2002 supra |
| | ΔpolyS/T/V | Δ175-237 | Itoh et al., 2002 supra |
| T. aestivum | | | |
| Rht-B1a | B1b | Q64stop → Δ1-67 | Peng et al., 1999 supra |
| Rht-D1a | D1b | E64stop → Δ1-67 | Peng et al., 1999 supra |
| H. vulgare | | | |
| SLN1 | sln1d | G46E | Chandler et al., Plant Physiology 129, |
| | | | p181-190, 2002 |
| A. thaliana | | | |
| GAI | gai | Δ27-43 | Peng et al., 1997 supra |
| RGA | rga-Δ17 | Δ44-60 | Dill et al., PNAS 98, p14162-67, |
| | | | 2001 |
| B. rapa | | | |
| RGA1 | brrga1-d | Q328R | Muangprom et al., 2005 supra |
| B. napus | | | |
| RGA1 | bzh | E546K | WO01/09356 |

The GA-sensitivity of DELLA protein can be measured by e.g. (over)expressing the protein in a plant by methods known in the art and evaluating the effect on plant height or by (transiently) (over)expressing the protein in a plant or plant cell and evaluating breakdown of the protein in response to GA treatment, as described in e.g. Itoh et al., 2002 supra; Gubler et al., 2002 supra, Muangprom et al., 2005 supra. The GA-sensitivity of a plant comprising (alleles encoding) DELLA proteins can be evaluated by exogenously applying GA and determining the effect thereof on plant height, as e.g. described in Itoh et al., 2002 supra.

“Mutagenesis”, as used herein, refers to the process in which plant cells (e.g., a plurality of Brassica seeds or other parts, such as pollen, etc.) are subjected to a technique which induces mutations in the DNA of the cells, such as contact with a mutagenic agent, such as a chemical substance (such as ethylmethylsulfonate (EMS), ethylnitrosourea (ENU), etc.) or ionizing radiation (neutrons (such as in fast neutron mutagenesis, etc.), alpha rays, gamma rays (such as that supplied by a Cobalt 60 source), X-rays, UV-radiation, etc.), or a combination of two or more of these. Thus, the desired mutagenesis of one or more DELLA alleles may be accomplished by use of chemical means such as by contact of one or more plant tissues with ethylmethylsulfonate (EMS), ethylnitrosourea, etc., by the use of physical means such as x-ray, etc, or by gamma radiation, such as that supplied by a Cobalt 60 source. While mutations created by irradiation are often large deletions or other gross lesions such as translocations or complex rearrangements, mutations created by chemical mutagens are often more discrete lesions such as point mutations. For example, EMS alkylates guanine bases, which results in base mispairing: an alkylated guanine will pair with a thymine base, resulting primarily in G/C to A/T transitions. Following mutagenesis, Brassica plants are regenerated from the treated cells using known techniques. For instance, the resulting Brassica seeds may be planted in accordance with conventional growing procedures and following self-pollination seed is formed on the plants. Alternatively, doubled haploid plantlets may be extracted to immediately form homozygous plants, for example as described by Coventry et al. (1988, Manual for Microspore Culture Technique for Brassica napus. Dep. Crop Sci. Techn. Bull. OAC Publication 0489. Univ. of Guelph, Guelph, Ontario, Canada). Additional seed that is formed as a result of such self-pollination in the present or a subsequent generation may be harvested and screened for the presence of mutant DELLA alleles. Several techniques are known to screen for specific mutant alleles, e.g., Deleteagene™ (Delete-a-gene; Li et al., 2001, Plant J 27: 235-242) uses polymerase chain reaction (PCR) assays to screen for deletion mutants generated by fast neutron mutagenesis, TILLING (targeted induced local lesions in genomes; McCallum et al., 2000, Nat Biotechnol 18:455-457) identifies EMS-induced point mutations, etc. Additional techniques to screen for the presence of specific mutant DELLA alleles are described in the Examples below.

A “(molecular) marker” as used herein refers to a measurable, genetic characteristic with a fixed position in the genome, which is normally inherited in a Mendelian fashion, and which can be used for mapping of a trait of interest. The nature of the marker is dependent on the molecular analysis used and can be detected at the DNA, RNA or protein level. Genetic mapping can be performed using molecular markers such as, but not limited to, RFLP (restriction fragment length polymorphisms; Botstein et al. (1980), Am J Hum Genet. 32:314-331; Tanksley et al. (1989), Bio/Technology 7:257-263), RAPD (random amplified polymorphic DNA; Williams ef a/. (1990), NAR 18:6531-6535), AFLP (Amplified Fragment Length Polymorphism; Vos et al. (1995) NAR 23:4407-4414), SNPs or microsatellites (also termed SSR's; Tautz et al. (1989), NAR 17:6463-6471), Invader™ technology, (as described e.g. in U.S. Pat. No. 5,985,557 “Invasive Cleavage of Nucleic Acids”, 6,001,567 “Detection of Nucleic Acid sequences by Invader Directed Cleavage, incorporated herein by reference), PCR or RT-PCR-based detection methods, such as TaqMan® (Applied Biosystems), or other detection methods, such as SNPlex, and the like.

A molecular marker is said to be “linked” to a gene or locus, if the marker and the gene or locus have a greater association in inheritance than would be expected from independent assortment, i.e., the marker and the locus co-segregate in a segregating population and are located on the same chromosome. “Linkage” refers to the genetic distance of the marker to the gene or locus (or two loci or two markers to each other). Closer is the linkage, smaller is the likelihood of a recombination event between the marker and the gene or locus. Genetic distance (map distance) is calculated from recombination frequencies and is expressed in centi Morgans (cM) (Kosambi (1944), Ann. Eugenet. 12:172-175).

Whenever reference to a “plant” or “plants” according to the invention is made, it is understood that also plant parts (cells, tissues or organs, seed pods, seeds, severed parts such as roots, leaves, flowers, pollen, etc.), progeny of the plants which retain the distinguishing characteristics of the parents, such as seed obtained by selfing or crossing, e.g. hybrid seed (obtained by crossing two inbred parental lines), hybrid plants and plant parts derived there from are encompassed herein, unless otherwise indicated.

“Crop plant” refers to plant species cultivated as a crop, such as, but not limited to, a Brassica plant, including Brassica napus (AACC, 2n=38), Brassica juncea (AABB, 2n=36), Brassica carinata (BBCC, 2n=34), Brassica rapa (syn. B. campestris) (AA, 2n=20), Brassica oleracea (CC, 2n=18) or Brassica nigra (BB, 2n=16). The definition does not encompass weeds, such as Arabidopsis thaliana.

A “variety” is used herein in conformity with the UPOV convention and refers to a plant grouping within a single botanical taxon of the lowest known rank, which grouping can be defined by the expression of the characteristics resulting from a given genotype or combination of genotypes, can be distinguished from any other plant grouping by the expression of at least one of the said characteristics and is considered as a unit with regard to its suitability for being propagated unchanged (stable).

As used herein, the term “non-naturally occurring” or “cultivated” when used in reference to a plant, means a plant with a genome that has been modified by man. A transgenic plant, for example, is a non-naturally occurring plant that contains an exogenous nucleic acid molecule, e.g., a chimeric gene comprising a transcribed region which when transcribed yields a biologically active RNA molecule that is translated into a protein, such as a DELLA protein according to the invention, and, therefore, has been genetically modified by man. In addition, a plant that contains a mutation in an endogenous gene, for example, a mutation in an endogenous DELLA gene, (e.g. in a regulatory element or in the coding sequence) as a result of an exposure to a mutagenic agent is also considered a non-natural plant, since it has been genetically modified by man. Furthermore, a plant of a particular species, such as Brassica napus, that contains a mutation in an endogenous gene, for example, in an endogenous DELLA gene, that in nature does not occur in that particular plant species, as a result of, for example, directed breeding processes, such as marker-assisted breeding and selection or introgression, with a plant of the same or another species, such as Brassica juncea or rapa, of that plant is also considered a non-naturally occurring plant. In contrast, a plant containing only spontaneous or naturally occurring mutations, i.e. a plant that has not been genetically modified by man, is not a “non-naturally occurring plant” as defined herein. One skilled in the art understands that, while a non-naturally occurring plant typically has a nucleotide sequence that is altered as compared to a naturally occurring plant, a non-naturally occurring plant also can be genetically modified by man without altering its nucleotide sequence, for example, by modifying its methylation pattern.

As used herein, “an agronomically suitable plant development” refers to a development of the plant, in particular an oilseed rape plant, which does not adversely affect its performance under normal agricultural practices, more specifically its establishment in the field, vigor, flowering time, height, maturation, yield, disease resistance, resistance to pod shattering, oil content and composition etc. Thus, lines with significantly increased lodging resistance with agronomically suitable plant development have lodging resistance that has increased as compared to other plants while maintaining a similar establishment in the field, vigor, flowering time, height, maturation, yield, disease resistance, resistance to pod shattering, oil content and composition, etc.

As used herein, “glucosinolates” are low molecular weight sulphur-containing glucosides that are produced and stored in almost all tissues of members of the Capparales, the most important member being the group of Crucifer plants. They are composed of two parts, a glycone moiety and a variable a glycone side chain derived from α-amino acids. Intake of large amounts of glucosinolates and their breakdown products is known to be toxic to animals and humans (WO97/016559). In Canada, the term “canola” describes oilseed rape with limited levels of glucosinolates and erucic acid in the harvested seeds, more specifically, after crushing, an air-dried meal containing less than 30 micromoles (pimp glucosinolates per gram of defatted (oil-free) meal (WO/1993/006714). Several assays are available for measuring both total and individual glucosinolates, e.g. alkenyl glucosinolates, in plants or parts thereof (e.g. Chavadej et al., Proc. Natl. Acad. Sci. USA 91, p2166-2170, 1994; Leonardo and Becker, Plant Breed. 117: p97-102, 1998; Wu et al., J. China Cereal Oil Assoc. 17: p59-62, 2002).

As used herein, “low glucosinolate content” refers to a glucosinolate content in the seed of lower than 30 μmol/g, preferably even lower, i.e. lower than 25 μmol/g, lower than 20 μmol/g, lower than 15 μmol/g of the oil-free meal.

As used herein, “the nucleotide sequence of SEQ ID NO:. Z from position X to position Y” indicates the nucleotide sequence including both nucleotide endpoints.

The term “comprising” is to be interpreted as specifying the presence of the stated parts, steps or components, but does not exclude the presence of one or more additional parts, steps or components. A plant comprising a certain trait may thus comprise additional traits.

It is understood that when referring to a word in the singular (e.g. plant or root), the plural is also included herein (e.g. a plurality of plants, a plurality of roots). Thus, reference to an element by the indefinite article “a” or “an” does not exclude the possibility that more than one of the element is present, unless the context clearly requires that there be one and only one of the elements. The indefinite article “a” or “an” thus usually means “at least one”.

### DETAILED DESCRIPTION

A mutagenized population of Brassica napus plants was evaluated for plants with a dwarf phenotype, i.e. reduced height. One such dwarf plant, which was named dwarf2 (dwf2) could be identified bearing a point mutation in the RGA1 genomic DNA resulting in a proline (P) to leucine (L) amino acid substitution (missense mutation) corresponding to amino acid position 91 in the B. napus RGA1 protein (SEQ ID NO: 3). When backcrossing this dwf2 allele into an elite B. napus line, the dwarf phenotype was stably maintained while the negative effect on yield that is usually associated with this type of mutations in Brassica species was not observed. Further, glucosinolate levels in seed from these plants appeared to be much lower than when a similar B. rapa RGA1 dwarf allele brrga1 was backcrossed into the same B. napus elite line.

This P91L substitution occurs in the VHYNP motif/conserved region II (indicated in FIG. 1), which when deleted, is known to confer a dwarf phenotype in maize and rice. Peng et al. (1999) describe two dominant maize severe dwarf mutants, mlp and 2038, comprising a deletion in the D8 DELLA protein of amino acids 1-105 and 87-98 respectively. Itoh et al. (2002) describe a similar severe dwarf mutant in rice, corresponding to a deletion of amino acids 87-104 of the SLR1DELLA protein. Based on these data, the smallest region to be deleted in order to confer a dwarf phenotype would correspond to amino acids 92-103 of the B. napus RGA1 protein (boxed in FIG. 1). The inventors have now found that a modification of at least one of the amino acids in this minimal region is sufficient to confer a dwarf phenotype to Brassica plants expressing this protein variant.

Thus, in a first embodiment the invention provides a plant comprising in its genome at least one mutant allele of a DELLA gene, said mutant allele encoding a dwarfing mutant DELLA protein comprising the amino acid sequence of SEQ ID NO. 1, characterized in that at least one amino acid of said sequence has been modified.

As used herein “modified” or “modification” refers to an alteration in an amino acid sequence, which can comprise both a substitution of one or more amino acids or a deletion or insertion of one or more amino acids. Whether a particular amino acid substitution, deletion or insertion results in a DELLA protein that confers a dwarf phenotype to the plant in which it is expressed and/or a DELLA protein that is GA-insensitive can be tested via methods as described above.

In one embodiment, the modification may involve a modification of the amino acid P (proline) of the amino acid sequence of SEQ ID NO. 1. The amino acid P may be substituted by any other amino acid or may be deleted. In another embodiment, the amino acid P may be modified into L (Leucine).

It will be understood that the plants according to the invention are significantly reduced in height and/or are significantly more resistant to lodging when compared to plants not comprising the mutant dwarfing DELLA allele. Preferably, the plants of the invention do not have a reduced yield when compared tot plants not comprising the mutant dwarfing DELLA allele and may even have improved yield due to less harvest loss. The plants of the invention also preferably maintain an agronomically suitable development and low glucosinolate content in the seed.

The invention also provides nucleic acid sequences representing dwarfing DELLA alleles. Nucleic acid sequences of wild type DELLA alleles are represented in the sequence listing, while the mutants of these sequences, and of sequences essentially similar to these, are described herein below and in the Examples, with reference to the wild type DELLA sequences.

“DELLA nucleic acid sequences” or “DELLA variant nucleic acid sequences” according to the invention are nucleic acid sequences encoding an amino acid sequence having at least 50%, at least 60%, at least 70%, at least 75%, at least 80%, at least 85%, at least 90%, at least 95%, 98%, 99% or 100% sequence identity with SEQ ID NO. 3, SEQ ID NO. 5, SEQ ID NO. 7 or SEQ ID NO. 9 or nucleic acid sequences having at least 50%, at least 60%, at least 70%, at least 75%, at least 80%, at least 85%, at least 90%, at least 95%, 98%, 99% or 100% sequence identity with SEQ ID NO. 2, SEQ ID NO. 4, SEQ ID NO. 6 or SEQ ID NO. 8. These nucleic acid sequences may also be referred to as being “essentially similar” or “essentially identical” to the DELLA sequences provided in the sequence listing.

Provided are nucleic acid sequences of dwarfing mutant DELLA alleles (comprising one or more mutations which result in an alteration in the amino acid sequence of the corresponding DELLA protein when compared to the wild-type protein) of DELLA genes. Such mutant alleles (referred to as della alleles) can be generated and/or identified using various known methods, as described further below, and are provided both in endogenous form and in isolated form. In one embodiment dwarfing mutant DELLA alleles (e.g. mutant RGA1 alleles), from Brassicaceae particularly from Brassica species, especially from Brassica napus, but also from other Brassica crop species are provided. For example, Brassica species comprising an A and/or a C genome may comprise different alleles of DELLA genes, which can be identified and transferred to another plant according to the invention. In addition, mutagenesis methods can be used to generate mutations in wild type DELLA alleles, thereby generating dwarfing mutant DELLA alleles for use according to the invention. Because specific DELLA alleles can be transferred from one plant to another by crossing and selection, in one embodiment the DELLA alleles are provided within a plant (i.e. endogenously), e.g. a Brassica plant, preferably a Brassica plant which can be crossed with Brassica napus or which can be used to make a “synthetic” Brassica napus plant. Hybridization between different Brassica species is described in the art, e.g., as referred to in Snowdon (2007, Chromosome research 15: 85-95). Interspecific hybridization can, for example, be used to transfer genes from, e.g., the C genome in B. napus (AACC) to the C genome in B. carinata (BBCC), or even from, e.g., the C genome in B. napus (AACC) to the B genome in B. juncea (AABB) (by the sporadic event of illegitimate recombination between their C and B genomes). “Resynthesized” or “synthetic” Brassica napus lines can be produced by crossing the original ancestors, B. oleracea (CC) and B. rapa (AA). Interspecific, and also intergeneric, incompatibility barriers can be successfully overcome in crosses between Brassica crop species and their relatives, e.g., by embryo rescue techniques or protoplast fusion (see e.g. Snowdon, above).

The nucleic acid molecules representing dwarfing mutant DELLA alleles may thus comprise one or more mutations, such as missense mutations or an insertion or deletion mutations, as is already described in detail above. Basically, any mutation which results in a protein comprising at least one amino acid insertion, deletion and/or substitution in SEQ ID NO. 1 relative to the wild type protein that leads to the formation of a DELLA protein which, when expressed in a plant, results in reduced height of that plant and/or increased lodging resistance of that plant (e.g. by creating a DELLA protein that acts constitutive repressor of GA-induced growth) corresponds to a dwarfing DELLA allele.

Thus in one embodiment, nucleic acid sequences comprising one or more of any of the types of mutations described above are provided. Any of the above mutant nucleic acid sequences are provided per se (in isolated form), as are plants and plant parts comprising such sequences endogenously.

Mutant DELLA alleles may be generated (for example induced by mutagenesis) and/or identified using a range of methods, which are conventional in the art, for example using PCR based methods to amplify part or all of the DELLA genomic or cDNA.

Following mutagenesis, plants are grown from the treated seeds, or regenerated from the treated cells using known techniques. For instance, mutagenized seeds may be planted in accordance with conventional growing procedures and following self-pollination seed is formed on the plants. Alternatively, doubled haploid plantlets may be extracted from treated microspore or pollen cells to immediately form homozygous plants, for example as described by Coventry et al. (1988, Manual for Microspore Culture Technique for Brassica napus. Dep. Crop Sci. Techn. Bull. OAC Publication 0489. Univ. of Guelph, Guelph, Ontario, Canada). Additional seed which is formed as a result of such self-pollination in the present or a subsequent generation may be harvested and screened for the presence of mutant DELLA alleles, using techniques which are conventional in the art, for example polymerase chain reaction (PCR) based techniques (amplification of the DELLA alleles) or hybridization based techniques, e.g. Southern blot analysis, BAC library screening, and the like, and/or direct sequencing of DELLA alleles. To screen for the presence of point mutations (so called Single Nucleotide Polymorphisms or SNPs) in mutant DELLA alleles, SNP detection methods conventional in the art can be used, for example oligoligation-based techniques, single base extension-based techniques, such as pyrosequencing, or techniques based on differences in restriction sites, such as TILLING.

The identified mutant alleles can then be sequenced and the sequence can be compared to the wild type allele to identify the mutation(s). Optionally, whether a mutant allele functions as a dwarf-inducing DELLA mutant allele can be tested as indicated above. Using this approach a plurality of mutant DELLA alleles (and plants comprising one or more of these) can be identified. The desired mutant alleles can then be transferred to other plants by crossing and selection methods as described further below.

Mutant DELLA alleles or plants (or plant parts) comprising mutant DELLA alleles can be identified or detected by method known in the art, such as direct sequencing, PCR based assays or hybridization based assays. Alternatively, methods can also be developed using the specific mutant DELLA allele specific sequence information provided herein. Such alternative detection methods include linear signal amplification detection methods based on invasive cleavage of particular nucleic acid structures, also known as Invader™ technology, (as described e.g. in U.S. Pat. No. 5,985,557 “Invasive Cleavage of Nucleic Acids”, 6,001,567 “Detection of Nucleic Acid sequences by Invader Directed Cleavage, incorporated herein by reference), RT-PCR-based detection methods, such as Taqman, or other detection methods, such as SNPlex.

It will be understood that the mutant DELLA alleles of the invention may also be used to generate transgenic plants. For example, the mutant allele may be transferred into a plant or plant cell via any method known in the art, such as transformation. The mutant allele may be used in combination with its own endogenous promoter or it may be used in a chimeric gene where it may be operably linked to a plant expressible promoter. Such chimeric gene may also comprise additional regulatory elements such as introns, transcription termination and polyadenylation sequences and the like.

Other species, varieties, breeding lines or wild accessions may be screened for other DELLA genes/alleles with the same or similar nucleotide sequence or variants thereof, as described above. In addition, it is understood that DELLA nucleotide sequences and variants thereof (or fragments of any of these) may be identified in silico, by screening nucleotide sequence databases for essentially similar sequences. In addition, it is understood that DELLA nucleotide sequences and variants thereof (or fragments of any of these) may be identified in silico, by screening nucleotide sequence databases for essentially similar sequences. Likewise, a nucleic acid sequence encoding a DELLA protein may be synthesized chemically.

The invention further provides a mutant dwarfing DELLA protein comprising the amino acid sequence of SEQ ID NO. 1, characterized in that at least one amino acid of said sequence has been modified.

Thus, the mutant DELLA proteins of the invention comprise one or more amino acid substitutions, insertions or deletions in the region corresponding to SEQ ID NO. 1 that result in the protein that, when expressed in a plant, confers a dwarf phenotype to that plant.

The amino acid sequence of mutant dwarfing DELLA proteins according to the invention, or variants thereof, are amino acid sequences having at least 50%, at least 60%, at least 70%, at least 75%, at least 80%, at least 85%, at least 90%, at least 95%, 98%, 99% or 100% sequence identity with SEQ ID NO. 3, SEQ ID NO. 5, SEQ ID NO. 7 or SEQ ID NO. 9. These amino acid sequences may also be referred to as being “essentially similar” or “essentially identical” to the DELLA sequences provided in the sequence listing. In one embodiment the mutant DELLA amino acid sequences are provided within a plant (i.e. endogenously). However, isolated DELLA amino acid sequences (e.g. isolated from the plant or made synthetically), as well as variants thereof and fragments of any of these are also provided herein.

In one embodiment, the modification of the amino acid sequence represented by SEQ ID NO. 1 may involve a modification of the amino acid P (proline). The amino acid P may be substituted by any other amino acid(s) or may be deleted. In another embodiment, the amino acid P may be modified into L (Leucine).

Other species, varieties, breeding lines or wild accessions may be screened for other DELLA proteins with the same amino acid sequences or variants thereof, as described above. In addition, it is understood that DELLA amino acid sequences and variants thereof (or fragments of any of these) may be identified in silico, by screening amino acid databases for essentially similar sequences

It is also an embodiment of the invention to provide plant cells containing the mutant DELLA alleles and proteins of the invention. Gametes, seeds, embryos, either zygotic or somatic, progeny or hybrids of plants comprising the mutant DELLA alleles of the present invention, which are produced by traditional breeding methods, are also included within the scope of the present invention.

The invention further provides Brassica seed comprising the RGA1 mutant allele dwf2, as comprised within seed having been deposited at the NCIMB Limited on Feb. 18, 2010, under accession number NCIMB 41697. Also provided are a Brassica plant, or a cell, part, seed or progeny thereof, obtained from the above described seeds, i.e. comprising the same RGA1 mutant allele dwf2 as the deposited seed.

The present invention also relates to the transfer of one or more specific mutant DELLA alleles from one plant to another plant, to the plants comprising those mutant DELLA alleles, the progeny obtained from these plants and to plant cells, plant parts, and plant seeds derived from these plants.

Thus, in one embodiment of the invention, a method for transferring at least one selected dwarfing mutant DELLA allele from one plant to another plant is provided comprising the steps of:
a. providing a first plant comprising the at least one mutant DELLA allele, as described above, or generating the first plant, as described above (wherein the first plant is homozygous or heterozygous for the at least one mutant DELLA allele);
b. crossing the first plant comprising the at least one mutant DELLA allele with a second plant not comprising the at least one mutant DELLA allele collecting F1 seeds from the cross (wherein the seeds are heterozygous for the mutant DELLA allele if the first plant was homozygous for that mutant DELLA allele, and wherein half of the seeds are heterozygous and half of the seeds are azygous for, i.e. do not comprise, the mutant DELLA allele if the first plant was heterozygous for that mutant DELLA allele);
   and optionally the further steps of;
c. identifying F1 plants comprising one or more selected mutant DELLA allele, as described above;
d. backcrossing F1 plants comprising at least one selected dwarfing mutant DELLA allele with the second plant not comprising the at least one selected mutant DELLA allele for one or more generations (x), collecting BCx seeds from the crosses; and
e. identifying in every generation BCx plants comprising the at least one selected mutant DELLA allele, as described above.

In another embodiment, the invention provides a method for producing a plant, in particular a Brassica crop plant, such as a Brassica napus plant, comprising at least one dwarfing mutant DELLA allele, but which preferably maintains an agronomically suitable development, is provided comprising transferring DELLA alleles according to the invention to one plant, as described above.

In yet another embodiment of the invention, a method for making a plant, in particular a Brassica crop plant, such as B. juncea, B. napus, B. rapa, B. carinata, B. oleracea and B. nigra, which is lodging resistant while maintaining an agronomically suitable development, is provided, comprising transferring DELLA alleles according to the invention into that plant, as described above.

Methods are also provided for increasing the lodging resistance of a plant and/or reducing the height of a plant comprising transferring at least one dwarfing mutant DELLA allele of the invention into the genomic DNA of said plant.

The invention also relates to the use of a dwarfing mutant DELLA allele of the invention to obtain plant with increased lodging resistance, in particular a Brassica crop plant, such as a Brassica napus plant.

The invention further relates to the use of a plant, in particular a Brassica crop plant, such as a Brassica napus plant, to produce seed comprising at least one dwarfing mutant DELLA allele or to produce a crop of oilseed rape, comprising at least one dwarfing mutant DELLA allele.

The invention additionally provides a process for producing dwarf Brassica plants and seeds thereof, comprising the step of crossing a plant consisting essentially of plant cells comprising a variant allele according to the invention with another plant or with itself, wherein the process may further comprise identifying or selecting progeny plants or seeds comprising the variant allele according to the invention, and harvesting seeds. The identification of the desired progeny plants may occur using molecular markers described herein.

Also provided is a method for producing oil or seed meal from the Brassica plants comprising the variant alleles according to the invention, comprising the steps known in the art for extracting and processing oil from seeds of oilseedrape plant.

The invention also provides a process for increasing the lodging resistance, and consequently the harvestable seeds comprising the steps of obtaining Brassica plants comprising a mutant allele as described elsewhere in the this application, and planting said Brassica plants in a field.

Further provided are methods for increasing lodging resistance or the amount of harvestable seeds in Brassica plants, comprising introducing a variant allele as described elsewhere in this application, into the genome of the Brassica plants.

It is understood that the lodging resistance and/or the yield of the plants of the invention, particularly dwf2 plants, can be further be improved (via an additive or synergistic effect with the dwarfing DELLA allele/protein) by treatment with certain (combinations of) plant growth regulators (PGRs). PGRs can be any compound or mixtures thereof which can influence germination, growth, ripening/maturation or development of plants, fruits or progeny. Plant growth regulators can be divided into different subclasses as exemplified herein.

anti-auxins, for example clofibrin [2-(4-chlorphenoxy)-2-methylpropanoic acid] and 2,3,5-tri-iodine benzoic acid;

auxine, for example 4-CPA (4-chlorphenoxy acetic acid), 2,4-D (2,4-dichlorphenoxy acetic acid), 2,4-DB[4-(2,4-dichlorphenoxy)butyric acid], 2,4-DEP {tris[2-(2,4-dichlorphenoxy)ethyl]phosphite}, dichlorprop, fenoprop, IAA (β-indole acetic acid), IBA (4-indol-3-yl butyric acid), naphthalin acetamide, α-naphthalin acetic acid, 1-naphthol, naphthoxy acetic acid, potassium naphthenate, sodium naphthenate, 2,4,5-T [(2,4,5-trichlorphenoxy)acetic acid];

cytokinine, for example 2iP [N-(3-methyl but-2-enyl)-1H-purin-6-amine], benzyladenine, kinetin, zeatin;

defoliants, for example calcium cyanamide, dimethipin, endothal, ethephon, merphos, metoxuron, pentachlorphenol, thidiazuron, tribufos;

ethylene inhibitors, for example aviglycine, aviglycine-hydrochloride, 1-methyl cyclopropene;

ethylene generators, for example ACC (1-amino cyclopropane carboxylic acid), etacelasil, ethephon, glyoxime;

gibberellins, for example gibberellins A1, A4, A7, gibberellic acid (=gibberellin A3);

growth inhibitors, for example abscisic acid, ancymidol, butralin, carbaryl, chlorphonium or the corresponding chloride, chlorpropham, dikegulac, sodium dikegulac, flumetralin, fluoridamid, fosamine, glyphosine, isopyrimol, jasmonic acid, maleic acid hydrazide or the potassium salt thereof, mepiquat or the corresponding chloride, piproctanyl or the corresponding bromide, pro-hydrojasmon, propham, 2,3,5-tri-iod benzoic acid;

morphactines, for example chlorfluren, chlorflurenol, chlorflurenol-methyl, dichlorflurenol, flurenol;

growth retardants or modifiers, for example chlormequat, chlormequat-chloride, daminozide, Flurprimidol, mefluidide, mefluidide-diolamine, paclobutrazol, cyproconazole, tetcyclacis, uniconazole, uniconazole-P;

growth stimulators, for example brassinosteroids (e.g. brassinolide), forchlorfenuron, hymexazol, 2-amino-6-oxypurin-derivative, indolinon derivatives, 3,4-disubstituted maleimide derivatives and azepinon-derivatives;

non-classified PGRs, for example benzofluor, buminafos, carvone, ciobutide, clofencet, potassium clofence, cloxyfonac, sodium cloxyfonac, cyclanilide, cycloheximide, epocholeone, ethychlozate, ethylene, fenridazon, heptopargil, holosulf, inabenfide, karetazan, lead arsenate, methasulfocarb, prohexadione, calcium prohexadione, pydanon, sintofen, triapenthenol, trinexapac and trinexapac-ethyl;

and other PGRs, for example 2,6-diisopropylnaphthalin, cloprop, 1-naphthyl acetic acidethylester, isoprothiolane, MCPB-ethyl [4-(4-chlor-o-tolyloxy)butyric acid ethyl ester], N-acetylthiazolidin-4-carbonic acid, n-decanol, pelargonic acid, N-phenylphthaliminic acid, tecnazene, triacontanol, 2,3-dihydro-5,6-diphenyl-1,4-oxathiin, 2-cyano-3-(2,4-dichlorophenyl)acrylic acid, 2-hydrazinoethanol, alorac, amidochlor, BTS 44584 [dimethyl(4-piperidinocarbonyloxy-2,5-xylyl)-sulfonium-toluene-4-sulfonate], chloramben, chlorfluren, chlorfluren-methyl, dicamba-methyl, dichlorflurenol, dichlorflurenol-methyl, dimexano, etacelasil, hexafluor acetone-trihydrate, N-(2-ethyl-2H-pyrazol-3-yl)-N′-phenyl-urea, N-m-tolylphthalaminis acid, N-pyrrolidinosuccinaminic acid, 3-tert-butyl phenoxy acetic acid propyl ester, pydanon, sodium (Z)-3-chloracrylate.

Preferred embodiments are chlormequat, chlormequat-chlorid, cyclanilide, dimethipin, ethephon, flumetralin, flurprimidol, inabenfide, mepiquat, mepiquat chloride, 1-methyl cyclopropene, paclobutrazol, prohexadion-calcium, pro-hydrojasmon, tribufos, thidiazuron, trinexapac, trinexapac-ethyl or uniconazol.

Particularly preferred are trinexapac-ethyl, chlormequat-chlorid and paclobutrazol as PGRs to be used with the plants of the invention, particularly dwf2 plants.

The plants of the invention or seeds thereof may be treated with herbicides, such as Clopyralid, Diclofop, Fluazifop, Glufosinate, Glyphosate, Metazachlor, Trifluralin Ethametsulfuron, Quinmerac, Quizalofop, Clethodim, Tepraloxydim

The plants of the invention or seeds thereof may also be treated with fungicides, such as Azoxystrobin, Bixafen, Boscalid, Carbendazim, Cyproconazole, Difenoconazole, Dimoxystrobin, Epoxiconazole, Fluazinam, Fluopyram, Fluoxastrobin, Flusilazole, Fluxapyroxad, Iprodione, Isopyrazam, Mepiquat-chloride, Metconazole, Metominostrobin, Paclobutrazole, Penthiopyrad, Picoxystrobin, Prochloraz, Prothioconazole, Pyraclostrobin, Tebuconazole, Thiophanate-methyl, Trifloxystrobin, Vinclozolin.

The plants of the invention or seeds thereof may also be treated with insecticides, such as Carbofuran, Thiacloprid, Deltamethrin, Imidacloprid, Clothianidin, Thiamethoxam, Acetamiprid, Dinetofuran, β-Cyfluthrin, gamma and lambda Cyhalothrin, tau-Fluvaleriate, Ethiprole, Spinosad, Spinotoram, Flubendiamide, Rynaxypyr, Cyazypyr, 4-[[(6-Chlorpyridin-3-yl)methyl] (2,2-difluorethyl)amino]furan-2(5H)-on.

The invention thus also relates to a process of applying a herbicide or insecticide or fungicide, particularly a herbicide or insecticide or fungicide of the above mentioned lists on a plant or seed of a plant comprising any variant allele as elsewhere described in this application.

The following non-limiting examples describe the characteristics of oilseed rape plants obtained in accordance with the invention. Unless otherwise stated, all molecular and recombinant DNA techniques are carried out according to standard protocols as described in Sambrook et al. (1989) Molecular Cloning: A Laboratory Manual, Second Edition, Cold Spring Harbour Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R.D.D. Croy published by BIOS Scientific Publications Ltd (UK) and Blackwell Scientific Publications, UK.

In the description and examples, reference is made to the following sequences:

### Sequences

SEQ ID NO. 1: Conserved region II consensus sequence, based on an alignment of the amino acid sequences of B. napus RGA1, B. rapa RGA1, A. thaliana RGA and GAI, maize D8 and D9, rice SLR1, wheat Rht and barley SLN1 proteins.

SEQ ID NO. 2: Genomic DNA/coding sequence of the RGA1 gene from Brassica napus.

SEQ ID NO. 3: Amino acid sequence of the RGA1 protein from Brassica napus.

SEQ ID NO. 4: Genomic DNA/coding sequence of the RGA1 gene from Brassica rapa.

SEQ ID NO. 5: Amino acid sequence of the RGA1 protein from Brassica rapa.

SEQ ID NO. 6: Genomic DNA/coding sequence of the RGA gene from Arabidopsis thaliana.

SEQ ID NO. 7: Amino acid sequence of the RGA protein from Arabidopsis thaliana

SEQ ID NO. 8: Genomic DNA/coding sequence of the GAI gene from Arabidopsis thaliana.

SEQ ID NO. 9: Amino acid sequence of the GAI protein from Arabidopsis thaliana.

### EXAMPLES

### Example 1

### Generation of Dwarfed Brassica Plants by Random Mutagenesis

A mutagenized Brassica napus population was generated as follows:

30,000 seeds from an elite spring oilseed rape breeding line (M0 seeds) were preimbibed for two hours on wet filter paper in deionized or distilled water. Half of the seeds were exposed to 0.8% EMS and half to 1% EMS (Sigma: M0880) and incubated for 4 hours.

The mutagenized seeds (M1 seeds) were rinsed 3 times and dried in a fume hood overnight. 30,000 M1 plants were grown in soil and selfed to generate M2 seeds. M2 seeds were harvested for each individual M1 plant.

5000 M2 plants, derived from different M1 plants, were grown and analyzed for the presence of plants with a dwarf phenotype (i.e. having a reduced height).

Dwarfed plants were identified in the mutant population with a similar phenotype as B. napus plants in which the Brrga1-d allele had been backcrossed, but somewhat stronger (i.e. more reduced height). The dwarf phenotype of the identified plants is semi-dominant, i.e. the heterozygotes display an intermediate dwarf phenotype when compared to the homozygous mutants and the wild-type segregants.

### Example 2

### Identification of Dwarf Mutant Alleles

Of the identified dwarf plants, DNA samples were prepared from leaf samples of each individual M2 plant according to the CTAB method (Doyle and Doyle, 1987, Phytochemistry Bulletin 19:11-15).

To identify the genomic position of the EMS mutations linked to the dwarf phenotype, BSA genetic mapping analysis was performed. The dwarf mutation termed dwf2 was found to be located on chromosome N06, at 109.99 cM, which is close to the reported position (R6) of the Brrga1 gene (Muangprom and Osborn, Theor Appl Genet. 108, p1378-1384, 2004; Muangprom et al., 2005 supra).

To confirm that RGA1 is indeed the causative gene of the dwf2 mutation, the RGA1 gene of the dwf2 mutant was screened by direct sequencing using standard sequencing techniques (Agowa) and the sequences were analyzed for the presence of the point mutations using the NovoSNP software (VIB Antwerp).

The RGA1 allele of dwf2 was found to comprise a C to T mutation at position 272 of the genomic/coding sequence as compared tot the wild-type RGA1 sequences (SEQ ID NO: 2), coding for an amino acid sequence comprising a Pro to Leu substitution at position 91, as compared to the wild-type RGA1 amino acid sequence (SEQ ID NO: 3).

Seeds comprising the dwf2 allele (designated 07 MBBN000265) have been deposited at the NCIMB Limited (Ferguson Building, Craibstone Estate, Bucksburn, Aberdeen, Scotland, AB21 9YA, UK) on Feb. 18, 2010, under accession number NCIMB 41697.

In conclusion, the above examples show how dwarfed Brassica plants can be generated and their corresponding mutant alleles can be identified. Also, plant material comprising such mutant alleles can be used to transfer selected mutant alleles into another plant, as described in the following examples.

### Example 3

### Identification of a Brassica Plant Comprising a Mutant RGA1 Allele

Brassica plants comprising the mutation in the RGA1 gene identified in Example 1 and 2 were identified as follows:

For each mutant RGA1 allele identified in the DNA sample of an M2 plant, at least 48 M2 plants derived from the same M1 plant as the M2 plant comprising the RGA1 mutation were grown and DNA samples were prepared from leaf samples of each individual M2 plant.

The DNA samples were screened for the presence of the identified RGA1 point mutations as described above in Example 2.

Heterozygous and homozygous (as determined based on the electropherograms) M2 plants comprising the same mutation were selfed and backcrossed, and BC1 seeds were harvested.

### Example 4

### Detection and/or Transfer of Mutant RGA1 Alleles into (Elite) Brassica Lines

The identified mutant RGA1 allele dwf2 was transferred into an (elite) Brassica napus breeding line by the following method: A plant containing the mutant dwf2 allele (donor plant), was crossed with an (elite) Brassica line (elite parent/recurrent parent) or variety lacking the mutant RGA1 gene. The following introgression scheme was used (+=wildtype allele, −=mutant allele):

Initial cross: −/− (donor plant) × +/+ (elite parent)
F1 plant: +/−
BC1 cross: +/− × +/+ (recurrent parent)
BC1 plants: 50% +/− and 50% +/+
The 50% +/− were selected.
BC2 cross: +/− (BC1 plant) × +/+ (recurrent parent)
BC2 plants: 50% +/− and 50% +/+
The 50% +/− were selected.
Backcrossing is repeated until BC3 to BC5
BC3-5 plants: 50% +/− and 50% +/+
The 50% +/− were selected.
BC3-5 S1 cross: +/− × +/−
BC3-5 S1 plants: 25% +/+, 50% +/− and 25% −/−
Individual BC3-5 S1 or BC3-5 S2 +/+, +/− and −/− plants were selected.

Similarly, the B. rapa RGA1 mutant allele Brrga1-d (Muangprom et al., 2005 supra) was transferred into the same (elite) B. napus breeding line.

To select for plants with a specific RGA1 genotype (+/+, +/− or −/−), direct sequencing by standard sequencing techniques known in the art, such as those described in Example 2, can be used. Alternatively, they can be selected using molecular markers (e.g. AFLP, PCR, Invader™, TaqMan® and the like) for mutant and wild-type RGA1 alleles.

### Example 5

### Evaluation of the Dwf2 and Brrga1 Mutant Phenotypes

The BC5-S2 Dwf2 plants generated in Example 4 were grown in the field on three locations A, B and C in both Belgium and Canada (3 plots per location) and subsequently analyzed for height, lodging resistance and yield. Lodging was evaluated on a visual scale of 1-9, whereby 9 indicates no lodging (all plants stand up straight) and 1 indicates severe lodging (all plants flattened). Furthermore, glucosinolate content in the oil-free meal of the seed obtained from these plants is measured with a NIRSystems 6500 near-infrared spectrophotometer at a wavelength range of 1098 to 2492 nm. The average results are presented in table 2.

TABLE 2
Field trial results BC5S2 dwf2 plants.

| | Height | | | Lodging | | | Yield | | | Gluc | | |
|---|---|---|---|---|---|---|---|---|---|---|---|---|
| | A | B | C | A | B | C | A | B | C | A | B | C |
| | Belgium | | | | | | | | | | | |
| −/− | 65 | 63 | 64 | 9.0 | 9.0 | 9.0 | 2680 | 2707 | 2693 | 15.1 | 16.2 | 15.6 |
| +/− | 92 | 91 | 91 | 9.0 | 9.0 | 9.0 | 2645 | 2806 | 2725 | 15.2 | 16.0 | 15.6 |
| +/+ | 128 | 124 | 126 | 6.0 | 8.0 | 7.0 | 2673 | 2570 | 2622 | 14.3 | 16.1 | 15.2 |
| control | 131 | 123 | 127 | 5.5 | 7.5 | 6.5 | 2755 | 2654 | 2704 | 14.8 | 15.8 | 15.3 |
| CV | 5.0 | 3.0 | 4.0 | 16.8 | 6.6 | 13.7 | 21.0 | 6.0 | 15.0 | 4.8 | 4.3 | 4.5 |
| LSD | 6.0 | 4.0 | 4.0 | 1.6 | 0.7 | 0.9 | 730 | 214 | 335 | 0.9 | 0.9 | 0.6 |
| | Canada | | | | | | | | | | | |
| −/− | 65 | 64 | 60 | nd | nd | nd | 2471 | 2384 | 4169 | 8.4 | 9.5 | nd |
| +/− | 91 | 84 | 73 | nd | nd | nd | 3132 | 3307 | 4083 | 9.3 | 10.9 | nd |
| +/+ | 101 | 105 | 105 | nd | nd | nd | 3103 | 3239 | 3659 | 11.0 | 13.5 | nd |
| control | 101 | 110 | 100 | nd | nd | nd | 2970 | 3306 | 3883 | 11.7 | 12.5 | nd |
| CV | 5.6 | 4.5 | 4.7 | — | — | — | 7.2 | 4.8 | 8.9 | 20.5 | 3.0 | — |
| LSD | 5.8 | 4.6 | 4.4 | — | — | — | 232 | 162 | 420 | 2.5 | 0.0 | — |

Height: plant height at end of flowering (cm), Lodging: lodging at maturity (1 = flat, 9 = straight), Yield: seed yield per plot (g), Gluc: total glucosinolate content in dry seed (μmol/g), CV: coefficient of variation, LSD: Least Significant Distance (p < 0.05), nd: not determined.

It can be seen that the dwf2 allele influenced plant height in a dose dependent manner, allowing easy discrimination between plants of various genotypes (−/−, +/− and +/+). By contrast, lodging was equally reduced in homozygous and heterozygous dwf2 plants, indicating that a single dwf2 allele is already sufficient to obtain plants with increased lodging resistance. Further, no significant difference (i.e. no decrease) in yield was observed between homozygous and heterozygous mutants on the one hand and wild-type segregants and the elite control on the other hand. Glucosinolate content of the seed was always well below the 30 micromoles per gram threshold required for canola.

Thus, in contrast to the previously identified brrga1-d and bzh alleles, which are associated with lower seed yield in inbred lines (Muangprom et al., 2006 supra) and hybrids (“Avenir”), even in homozygous form in inbred lines, the present dwf2 allele already performs equally well in terms of yield as the elite control line. It is expected that seed yield will further improve in hybrid crosses with the dwf2 allele.

To evaluate the effect of the B. rapa background on seed oil composition in backcrosses with the brrga1-d allele, seeds of various brrga1 and dwf2 backcrosses of example 4 were sown in the greenhouse and the seeds obtained from the plants grown from those seeds were analyzed for glucosinolate content (Table 3).

TABLE 3
Glucosinolate content in seed oil of brrga1-d BC5S2 and dwf2 BC2S3
homozygous plants (−/−) and wild-type segregants (+/+), and of individual
progeny (25% +/+, 50% +/−, 25% −/−) of brrga1-d BC9 plants (+/−)

| allele | backcross | genotype | gluc |
|---|---|---|---|
| brrga1-d | BC5S2 | −/− | 36.1 |
| | | +/+ | 36.4 |
| dwf2 | BC2S3 | −/− | 19.3 |
| | | +/+ | 17.9 |
| brrga1-d | BC9 | 25% −/− | 30.1 |
| | | 50% +/− | 21.9 |
| | | 25% +/+ | 14.3 |
| | | | 30.2 |
| | | | 25.9 |
| | | | 20.8 |
| | | | 22.1 |

These results demonstrate that already in early backcrosses dwf2 mutants display a much more favorable glucosinolate seed oil content than brrga1-d mutants in more advanced backcrosses. The high glucosinolate content in the seed oil of BC5S2 brrga1-d mutants as well as wild-type segregants indicates that the high-glucosinolate phenotype originates from the rapa background. The more advanced backcross BC9 shows that high glucosinolate content still remains in most of the progeny (probably containing the brrga1-d allele, i.e. −/− and −/+ plants), indicating that this trait is still closely linked to the brrga1-d allele and it will probably not be possible to separate the brrga1-d and glucosinolate loci in even further backcrosses.

1. A Brassica plant comprising in its genome at least one mutant allele of a DELLA gene, said mutant allele encoding a dwarfing mutant DELLA protein comprising the amino acid sequence of SEQ ID NO. 1, characterized in that at least one amino acid of said sequence has been modified.

2. The plant of claim 1, wherein said at least one amino acid that has been modified is P.

3. The plant of claim 2, wherein said amino acid P has been modified to L.

4. The plant of any one of claims 1-3, wherein said protein comprises an amino acid sequence having at least 75% sequence identity to SEQ ID NO: 3, SEQ ID NO: 5 or SEQ ID NO: 7 or SEQ ID NO: 9.

5. The plant of any one of claims 1-4, which is more resistant to lodging and/or has a reduced height when compared to plants not comprising said mutant allele.

6. The plant of any one of claims 1-5, which is selected from the group consisting of B. juncea, B. napus, B. rapa, B. carinata, B. oleracea and B. nigra.

7. A plant cell, seed, or progeny of the plant of any one of claims 1-6.

8. A Brassica seed comprising a mutant RGA1 allele dwf2, as comprised within seed having been deposited at the NCIMB Limited on Feb. 18, 2010, under accession number NCIMB 41697.

9. A Brassica plant, or a cell, part, seed or progeny thereof, obtained from the seed of claim 8.

10. A dwarfing mutant DELLA allele encoding a dwarfing mutant DELLA protein comprising the amino acid sequence of SEQ ID NO. 1, characterized in that at least one amino acid of said sequence has been modified.

11. The mutant DELLA allele of claim 10, wherein said at least one amino acid that has been modified is P.

12. The mutant DELLA allele of claim 11, wherein said amino acid P has been modified to L.

13. The mutant DELLA allele of any one of claims 10-12, wherein said mutant DELLA protein comprises an amino acid sequence having at least 75% sequence identity to SEQ ID NO: 3, SEQ ID NO: 5, SEQ ID NO: 7 or SEQ ID NO: 9.

14. A dwarfing mutant DELLA protein comprising the amino acid sequence of SEQ ID NO. 1, characterized in that at least one amino acid of said sequence has been modified.

15. The protein of claim 14, wherein said at least one amino acid that has been modified is P.

16. The protein of claim 15, wherein said amino acid P has been modified to L.

17. The protein of any one of claims 14-16, comprising an amino acid sequence having at least 75% sequence identity to SEQ ID NO: 3, SEQ ID NO: 5, SEQ ID NO: 7 or SEQ ID NO: 9.

18. A method for transferring at least one selected dwarfing mutant DELLA allele from one plant to another plant comprising the steps of:
a) providing a first plant comprising at least one selected mutant DELLA allele of any one of claims 10-13 or generating a first plant comprising at least one selected mutant DELLA allele of any one of claims 10-13;
b) crossing the first plant with a second plant not comprising the at least one selected mutant DELLA allele and collecting F1 hybrid seeds from the cross;
and optionally the further steps of:
c) identifying F1 plants comprising the at least one selected mutant DELLA allele;
d) backcrossing F1 plants comprising the at least one selected mutant DELLA allele with the second plant not comprising the at least one selected mutant DELLA allele for at least one generation (x) and collecting BCx seeds from the crosses; and
e) identifying in every generation BCx plants comprising the at least one selected mutant DELLA allele.

19. A method for producing a plant of any one of claims 1-6 and 9 comprising transferring at least one mutant DELLA alleles from one plant to another plant, such as by the method of claim 18.

20. A method to increase the lodging resistance of a plant or reduce the height of a plant comprising transferring at least one dwarfing mutant DELLA allele of any one of claims 10-13 into the genomic DNA of said plant.

21. The method of any one of claims 18-20, wherein said plant is selected from the group consisting of B. juncea, B. napus, B. rapa, B. carinata, B. oleracea and B. nigra.

22. Use of a dwarfing mutant DELLA allele of any one of claims 10-13 to obtain a plant with reduced height or a plant with increased lodging resistance.

23. Use of the plant of any one of claim 1-6 or 9 to produce seed comprising at least one dwarfing mutant DELLA allele.

24. Use of the plant of any one of claim 1-6 or 9 to produce a crop of oilseed rape, comprising at least one dwarfing mutant DELLA allele.
